(19) United States
(12) Patent Application Publication
(10) Pub. No.: US 2015/0320723 A1
(43) Pub. Date: Nov. 12, 2015

(54) Use of Small Molecule Unfolder Protein Response Modulators to Treat Tumors With Active Sonic Hedgehog (SSH) Signaling Due To Smoothened (SMO) Mutation

(71) Applicant: ST. JUDE CHILDREN'S RESEARCH HOSPITAL, Memphis, TN (US)

(72) Inventors: Stacey Ogden, Memphis, TN (US); Suresh Marada, Cordova, TN (US)

(21) Appl. No.: 14/655,649

(22) PCT Filed: Jan. 6, 2014
(86) PCT No.: PCT/US14/10321

§ 371 Date: Jun. 25, 2015

Related U.S. Application Data
(60) Provisional application No. 61/749,631, filed on Jan. 7, 2013.

(51) Int. Cl. A61K 31/4166; A61K 31/416; A61K 31/395

(52) U.S. Cl. CPC A61K 31/4166; A61K 31/395; A61K 31/416

(57) ABSTRACT

The present invention relates to methods for treating tumors having SMO mutations as well as diagnosing tumors with SMO mutations. The invention relates to methods for the treatment of cancer with ER stress-inducing compounds or UPR inducing compounds. The ER stress-inducing compounds or UPR inducing compounds might fill a clinical need for additional methods of targeting the Hh pathway, either in frontline combination therapy or in salvage therapy for relapsed patients who develop resistance to the available SMO inhibitor and offer a significant advantage over SMO-specific small molecules. Because ER stress modulators and UPR inducing compounds exploit a cellular process that is distinct from the Hh signaling pathway, their efficacy should be unaltered by acquired SMO mutation.

### FIELD OF THE INVENTION

The present invention relates to methods for treating tumors having and active Sonic Hedgehog signaling pathway due to smoothened (SMO) mutations as well as diagnosing tumors with SMO mutations. The invention relates to methods for the treatment of cancer with endoplasmic reticulum (ER) stress-inducing compounds or unfolded protein response (UPR) inducing compounds. The ER stress-inducing compounds or UPR inducing compounds might fill a clinical need for additional methods of targeting the Hh pathway, either in frontline combination therapy or in salvage therapy for relapsed patients who develop resistance to the available SMO inhibitor and offer a significant advantage over SMO-specific small molecules. Because ER stress modulators and UPR inducing compounds exploit a cellular process that is distinct from the Hh signaling pathway, their efficacy should be unaltered by acquired SMO mutation.

### BACKGROUND OF THE INVENTION

Cancer is a genetic disease and in most cases involves mutations in one or more genes. There are believed to be around 30-40,000 genes in the human genome but only a handful of these genes have been shown to be involved in cancer. Although it is surmised that many more genes than have been presently identified will be found to be involved in cancer, progress in this area has remained slow despite the availability of molecular analytical techniques. This may be due to the varied structure and function of genes which have been identified to date which suggests that cancer genes can take many forms and have many different functions.

The Hedgehog signal transduction pathway, which is essential for pattern formation during development, is implicated as playing causative and survival roles in a range of human cancers. Accordingly, the requisite signal transducing component of the pathway, Smoothened, has revealed itself to be an efficacious therapeutic target. Despite clinical success, challenges remain in cases where oncogenic Hedgehog signaling is induced by somatic Smoothened mutation, and also in cases where tumors become resistant to Smoothened-specific antagonists. Therefore, there is a continuing need for methods for targeting oncogenic Smoothened signaling in cancer.

### SUMMARY OF THE INVENTION

The present invention relates to methods for treating tumors having SMO mutations as well as diagnosing tumors with SMO mutations. The invention relates to methods for the treatment of cancer with ER stress-inducing compounds or UPR inducing compounds. The ER stress-inducing compounds or UPR inducing compounds might fill a clinical need for additional methods of targeting the Hh pathway, either in frontline combination therapy or in salvage therapy for relapsed patients who develop resistance to the available SMO inhibitor and offer a significant advantage over SMO-specific small molecules. Because ER stress modulators and UPR inducing compounds exploit a cellular process that is distinct from the Hh signaling pathway, their efficacy should be unaltered by acquired SMO mutation.

In one embodiment, the invention relates to a method of treatment, comprising: administering an endoplasmic reticulum (ER) stressor compound to a subject having a cancer, wherein said cancer comprises a smoothened (SMO) mutation. In one embodiment, said cancer cells comprise Sonic Hedgehog (SHH)-driven tumors. In one embodiment, said cancer is selected form the group of cancers consisting of basal cell carcinoma, medulloblastoma, rhabdomyosarcoma, multiple myeloma and prostate cancer. In one embodiment, said treatment comprises administration of a drug selected from the group consisting of: 17-N-Allylamino-17-demethoxygeldanamycin (17-AAG KOS-953,Tanespimycin), 17-Dimethylaminoethylamino-17-demethoxygeldanamycin (17-DMAG, KOS-1022, Alvespimycin), 4-(6,6-dimethyl-4-oxo-3-(trifluoromethyl)-4,5,6,7-tetrahydroindazol-1-yl)-2-((1r,4r)-4-hydroxycyclohexylamino)benzamide (SNX-2112), and Eeyarestatin I (EerI). In one embodiment, said treatment comprises administration of a drug selected from the group consisting of: NPI-0052 (salinosporamide A), Carfilzomib (PR-171), PS-341, CEP-18770, Retaspimycin (IPI-504), PU-H71, Versipelostatin, (−)-epigallocatechin gallate (EGCG), Epidermal growth factor (EGF)-SubA, Irestatins, and Delta(9)-Tetrahydrocannabinol (THC).

In one embodiment, the invention relates to a method of treatment, comprising: administering an endoplasmic reticulum (ER) stressor stressor compound to a subject having a cancer that has become resistant to previous treatment with a hedgehog inhibitor, wherein said cancer comprises a smoothened (SMO) mutation. In one embodiment, said cancer is selected form the group of cancers consisting of basal cell carcinoma, rhabdomyosarcoma, multiple myeloma and prostate cancer. In one embodiment, said treatment comprises administration of a drug selected from the group consisting of: 17-N-Allylamino-17-demethoxygeldanamycin (17-AAG, KOS -953, Tanespimycin), 17-Dimethylaminoethylamino-17-demethoxygeldanamycin (17-DMAG, KOS-1022, Alvespimycin), 4-(6,6-dimethyl-4-oxo-3-(trifluoromethyl)-4,5,6,7-tetrahydroindazol-1-yl)-2-((1r,4r) -4-hydroxycyclohexylamino)benzamide (SNX-2112), and Eeyarestatin I (EerI). In one embodiment, said treatment comprises administration of a drug selected from the group consisting of: NPI-0052 (salinosporamide A), Carfilzomib (PR-171), PS-341, CEP-18770, Retaspimycin (IPI-504), PU-H71, Versipelostatin, (−)-epigallocatechin gallate (EGCG), Epidermal growth factor (EGF)-SubA, Irestatins, and Delta(9)-Tetrahydrocannabinol (THC).

In one embodiment, the invention relates to a method of treatment comprising: a) providing a sample of cancer from a subject; b) testing said sample to determine whether said cancer has a smoothened (SMO) mutation and whether tumor cells are sensitive to ER stressors ex vivo; and c) treating said subject with an endoplasmic reticulum (ER) stressor compound where said cancer comprises a smoothened (SMO) mutation. In one embodiment, said cancer is selected form the group of cancers consisting of basal cell carcinoma, rhabdomyosarcoma, medulloblastoma, multiple myeloma and prostate cancer. In one embodiment, said treatment comprises administration of a drug selected from the group consisting of: 17-N-Allylamino-17-demethoxygeldanamycin (17-AAG, KOS-953,Tanespimycin), 17-Dimethylaminoethylamino-17-demethoxygeldanamycin (17-DMAG, KOS-1022, Alvespimycin), 4-(6,6-dimethyl-4-oxo-3-(trifluoromethyl)-4,5,6,7-tetrahydroindazol-1-yl)-2-((1r,4r)-4-hydroxycyclohexylamino)benzamide (SNX-2112), and Eeyarestatin I (EerI). In one embodiment, said treatment comprises administration of a drug selected from the group consisting of: NPI-0052 (salinosporamide A), Carfilzomib (PR-171), PS-341, CEP-18770, Retaspimycin (IPI-504), PU-H71, Versipelostatin, (−)-epigallocatechin gallate (EGCG), Epidermal growth factor (EGF)-SubA, Irestatins, and Delta(9)-Tetrahydrocannabinol (THC).

In one embodiment, the invention relates to a method of treatment, comprising: administering an unfolded protein response (UPR) inducing compound to a subject having a cancer, wherein said cancer comprises a smoothened (SMO) mutation. In one embodiment, said cancer cells comprise Sonic Hedgehog (SHH)-driven tumors. In one embodiment, said cancer is selected form the group of cancers consisting of basal cell carcinoma, rhabdomyosarcoma, medulloblastoma, multiple myeloma and prostate cancer. In one embodiment, said treatment comprises administration of a drug selected from the group consisting of: 17-N-Allylamino-17-demethoxygeldanamycin (17-AAG, KOS-953,Tanespimycin), 17-Dimethylaminoethylamino-17-demethoxygeldanamycin (17-DMAG, KOS-1022, Alvespimycin), 4-(6,6-dimethyl-4-oxo-3-(trifluoromethyl)-4,5,6,7-tetrahydroindazol-1-yl)-2-((1r,4r)-4-hydroxycyclohexylamino)benzamide (SNX-2112), and Eeyarestatin I (EerI). In one embodiment, said treatment comprises administration of a drug selected from the group consisting of: NPI-0052 (salinosporamide A), Carfilzomib (PR-171), PS-341, CEP-18770, Retaspimycin (IPI-504), PU-H71, Versipelostatin, (−)-epigallocatechin gallate (EGCG), Epidermal growth factor (EGF)-SubA, Irestatins, and Delta(9)-Tetrahydrocannabinol (THC).

In one embodiment, the invention relates to a method of treatment, comprising: administering an unfolded protein response (UPR) inducing compound to a subject having a cancer that has become resistant to previous treatment with a hedgehog inhibitor, wherein said cancer comprises a smoothened (SMO) mutation. In one embodiment, said cancer is selected form the group of cancers consisting of basal cell carcinoma, leukemia, lymphoma, multiple myeloma and prostate cancer. In one embodiment, said treatment comprises administration of a drug selected from the group consisting of: 17-N-Allylamino-17-demethoxygeldanamycin (17-AAG, KOS-953,Tanespimycin), 17-Dimethylaminoethylamino-17-demethoxygeldanamycin (17-DMAG, KOS-1022, Alvespimycin), 4-(6,6-dimethyl-4-oxo-3-(trifluoromethyl)-4,5,6,7-tetrahydroindazol-1-yl)-2-((1r,4r)-4-hydroxycyclohexylamino)benzamide (SNX-2112), and Eeyarestatin I (EerI). In one embodiment, said treatment comprises administration of a drug selected from the group consisting of: NPI-0052 (salinosporamide A), Carfilzomib (PR-171), PS-341, CEP-18770, Retaspimycin (IPI-504), PU-H71, Versipelostatin, (−)-epigallocatechin gallate (EGCG), Epidermal growth factor (EGF)-SubA, Irestatins, and Delta(9)-Tetrahydrocannabinol (THC).

In one embodiment, the invention relates to a method of treatment comprising: a) providing a sample of cancer from a subject; b) testing said sample to determine whether said cancer has a smoothened (SMO) mutation; and c) treating said subject with an unfolded protein response (UPR) inducing compound where said cancer comprises a smoothened (SMO) mutation. In one embodiment, said cancer is selected from the group of cancers consisting of basal cell carcinoma, leukemia, lymphoma, multiple myeloma and prostate cancer. In one embodiment, said treatment comprises administration of a drug selected from the group consisting of: 17-N-Allylamino-17-demethoxygeldanamycin (17-AAG, KOS-953,Tanespimycin), 17-Dimethylaminoethylamino-17-demethoxygeldanamycin (17-DMAG, KOS-1022, Alvespimycin), 4-(6,6-dimethyl-4-oxo-3-(trifluoromethyl)-4,5,6,7-tetrahydroindazol-1-yl)-2-((1r,4)-4-hydroxycyclohexylamino)benzamide (SNX-2112), and Eeyarestatin I (EerI). In one embodiment, said treatment comprises administration of a drug selected from the group consisting of: NPI-0052 (salinosporamide A), Carfilzomib (PR-171), PS-341, CEP-18770, Retaspimycin (IPI-504), PU-H71, Versipelostatin, (−)-epigallocatechin gallate (EGCG), Epidermal growth factor (EGF)-SubA, Irestatins, and Delta(9)-Tetrahydrocannabinol (THC).

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “patient” or “subject” is used throughout the specification to describe an animal, generally a mammal and preferably a human, to whom treatment, including prophylactic treatment, with the compositions according to the present invention is provided. In certain embodiments, the patient or subject is a primate. Non-limiting examples of human subjects are adults, juveniles, infants and fetuses. For treatment of conditions or disease states, which are specific for a specific animal such as a human patient, the term patient refers to that specific animal.

As used herein, the terms “prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the terms “treat” and “treating” is used throughout the specification to describe a step or several steps of a process to achieve a goal. Additionally, as used herein, the terms “treat” and “treating” are not limited to the case where the subject or material (e.g. tissue, substrate, or patient) is cured and the disease is eradicated or material sterilized. Rather, the present invention also relates to treatment that merely reduces symptoms, improves (to some degree) and/or delays disease progression. It is not intended that the present invention be limited to instances wherein a disease, infection, or affliction is cured. It is sufficient that symptoms are reduced.

The present invention relates to the above-described compositions in “therapeutically effective amounts” or “pharmaceutically effective amounts”, which means that amount which, when administered to tissues is sufficient to effect such treatment for the disease, infection, or to ameliorate one or more symptoms of a disease or condition (e.g. ameliorate pain).

As used herein, the term “unfolded protein response (UPR)” is used throughout the specification to describe a compensatory process aimed at reducing the unfolded or misfolded protein burden of the ER to ameliorate endoplasmic reticulum stress and prevent stress-induced cell death. It is a stress response that has been found to be conserved between all mammalian species (conserved to yeast). The UPR is organized into three branches, each controlled by a unique upstream activator. The PERK branch triggers phosphorylation of elongation factor 2α to attenuate translation of nascent proteins bound for the ER (Harding et al., 1999 [1]). The ATF6 and IRE1α branches activate transcription factors that drive expression of UPR target genes involved in protein quality control and ER associated degradation (ERAD), a process that targets misfolded proteins for retro-translocation from the ER to the cytoplasm where they undergo proteasome-mediated degradation (McCracken & Brodsky, 1996 [2]; Tirasophon et al., 2000 [3]; Walter & Ron, 2011 [4]; and Yoshida et al., 1998 [5]). Although it is not necessary to understand the mechanism of an invention, it is believed that persistent ER stress that cannot be corrected by the UPR will eventually result in apoptosis (Walter & Ron, 2011 [4]). However, the exact mechanisms by which the UPR signals for induction of apoptosis under such conditions are not yet clear.

As used herein, the term “ER” or “Endoplasmic reticulum” is used throughout the specification to describe an organelle of cells in eukaryotic organisms that forms an interconnected network of tubules, vesicles, and cisternae. Rough endoplasmic reticulum are involved in the synthesis of proteins and is also a membrane factory for the cell, while smooth endoplasmic reticula are involved in the synthesis of lipids, including oils, phospholipids and steroids, metabolism of carbohydrates, regulation of calcium concentration and detoxification of drugs and poisons. Sarcoplasmic reticula solely regulate calcium levels. The luminal space within the double-layered nuclear membrane is continuous at points with the endoplasmic reticulum, whose membrane is continuous with the outer nuclear membrane (nuclear envelope).

As used herein, the term “Endoplasmic reticulum (ER)-associated protein degradation (ERAD)” is used throughout the specification to describe a cellular pathway which targets misfolded proteins of the endoplasmic reticulum for ubiquitination and subsequent degradation by a protein-degrading complex, called the proteasome. ERAD targets are selected by a quality control system within the ER lumen and are ultimately destroyed by the cytoplasmic ubiquitin-proteasome system (UPS). The spatial separation between substrate selection and degradation in ERAD requires substrate transport from the ER to the cytoplasm by a process termed retrotranslocation.

As used herein, the term “endoplasmic reticulum (ER) stressor” is used throughout the specification to describe a compound, substance, or condition that can cause altered glycosylation, prevention of ERAD, prevention of IREia kinase activity, etc. Compounds that enhance protein misfolding cause ER stress by “allowing” misfolded proteins to accumulate in the ER. Whereas the end result is the same, the processes are a little different . . . all UPR modulators will cause ER stress, but not all ER stressors will cause protein misfolding.

As used herein, the terms “hedgehog signaling pathway” or “sonic hedgehog signaling pathway” is used throughout the specification to describe one of the key regulators of animal development. Mammals have three Hedgehog homologues, of which Sonic hedgehog is the best studied. Inappropriate activation of the pathway has also been implicated in the development of some cancers. Mammalian Hedgehog proteins include Sonic Hedgehog (Shh), Indian Hedgehog (Ihh), and Desert Hedgehog (Dhh). Shh is expressed mainly in the epithelia in the tooth, hair, gut, bladder, urethra, vas deferens, and lung, Dhh is found in Schwann and Sertoli cell precursors and Ihh is expressed in gut and cartilage. Shh is the best-characterized Hedgehog protein. It is synthesized as a 45 kDa precursor protein, which is then auto-catalytically cleaved to generate a 20 kDa N-terminal fragment that is responsible for all Hh biological activity, and a 25 kDa C-terminal fragment that contains the auto-processing unit. The N-terminal fragment of Shh contains palmitic acid and cholesterol as two lipid tethers, which allow it to remain associated with the plasma membrane. The cholesterol moiety is believed to be responsible for directing Hedgehog traffic in the secretory cell.

Shh, a secreted morphogen, has been implicated in several embryonic developmental processes. It displays inductive, proliferative, neurotrophic, and neuroprotective properties. Shh often works inconcert with the Wnt signaling protein in setting embryonic patterns. The Wnt pathway uses β-catenin to transduce its signals to the nucleus; however, the Shh pathway utilizes a 155 amino acid protein, Cubitus interruptus (Ci155) in Drosophila or Gli in mammals. Shh signaling is known to occur through a receptor complex associating two membrane proteins, Patched (Ptc) and Smoothened (Smo). Ptc is a twelve-pass membrane protein that acts as a receptor and binds Hedgehog ligand; Smo is a seven-pass membrane protein that acts as a signal transducer. In this regard, Smo displays homology to G-protein-coupled receptors that are usually associated with heterotrimeric G-proteins and G-protein coupled receptor kinases. Patched inhibits Smo activity by an unknown mechanism, and seems to function enzymatically/catalytically [6]. Under these conditions, Ci is targeted for proteolysis, which generates a truncated 75-amino acid residues form (Ci75), which acts as a transcriptional repressor. In vertebrates three Gli proteins (Gli1, Gli2, and Gli3) have been reported and despite several homologous regions, including a DNA-binding domain with five C2-H2 zinc fingers and a C-terminal transcription activation domain, these proteins have distinct activities and are somewhat functionally equivalent. For example, work by Alex Joyner has shown that Gli2 knocked into the Gli1 locus can rescue Gli1 loss of function phenotypes in mice. Shh binding to Ptc removes the inhibitory effect on Smo and allows Ci/Gli to be stabilized in its full-length form, and to enter the nucleus and act as a transcriptional activator. Smo action is mediated through a protein complex containing the kinesin-like protein Costal2 (Cos2), the Ser/Thr kinase Fused (Fu) and Ci/Gli in Drosophila systems. Transcriptional activity of Ci/Gli is also regulated through its binding to Suppressor of Fu (Sufu), which is a negative regulator of hedgehog signaling in Drosophila as well as in vertebrates. It binds to all three Gli proteins with different affinities. Whereas it is known that Sufu is an essential regulator of Shh signaling in mammalian systems, the exact mechanisms by which the Shh activation signal is transduced by Smo to Sufu in mammalian systems is not yet clear. There is evidence that the Cos2 ortholog Kif7 may be involved.

Protein kinase A (PKA), casein kinase I (CKI) and glycogen synthase kinase 3B (GSK-3B) play a significant role in regulating hedgehog signaling process. They all bind to Cos2, and phosphorylate homologous domains on Ci/Gli and Smo. Phosphorylation of Ci by PKA, CKI and GSK-3B is shown to be essential for the efficient processing of Ci155 to its transcriptional repressor form, Ci75 Inhibition of any of these kinases can lead to Ci155 accumulation. The role of phosphorylation in the regulation of vertebrate Gli proteins has not yet been clearly defined, although PKA is shown to block vertebrate hedgehog signaling.

Shh signaling is required throughout embryonic development and is involved in the determination of cell fate and embryonic patterning during early vertebrate development. During the late stage of development, Shh is involved in the proper formation of a variety of tissues and organs and it functions with other signaling molecules, such as the fibroblast growth factors and bone morphogenetic protein, to mediate developmental processes. Mutations in any of the components of the Shh pathway can lead to congenital defects and diseases, including cancer. Disruption of Shh in humans leads to Holoprosencephaly (lack of development of forebrain in the embryo). Loss of patched, overexpression of Shh, and activating mutations of Gli have been reported in basal cell carcinomas. Amplification of Gli has also been shown in malignant gliomas and osteosarcoma. Mutations in Smo and Sufu have also been associated with the formation of sporadic basal cell carcinoma and medulloblastoma. Hence, the Shh pathway has become a potential target for drug development for the treatment of cancers and degenerative diseases.

As used herein, the terms “Hedgehog (HH)-driven tumors” is used throughout the specification to describe cancers or tumors wherein inappropriate SHH, IHH or DHH signaling is causative of the cancer or tumor formation.

HH signaling is inappropriately activated in the tumor, and affords a growth advantage to the tumor cell. As such, the tumor cell becomes addicted to the Hh pathway.

As used herein, the terms “hedgehog inhibitor” is used throughout the specification to describe a compound or substance which effectively reduces the activity of the hedgehog family signaling pathway, particularly in a tumor or cancer cell.

As used herein, the term “Drosophila” is used throughout the specification to describe a genus of small flies, belonging to the family Drosophilidae, whose members are often called “fruit flies.” One species of Drosophila in particular, D. melanogaster, has been heavily used in research in genetics and is a common model organism in developmental biology. The terms “fruit fly” and “Drosophila” are often used synonymously with D. melanogaster in modern biological literature. The Drosophila model system offers a powerful tool to dissect the HH signaling cascade because HH pathway components are tightly conserved from Drosophila to human, HH phenotypes are well characterized in Drosophila, and the power of Drosophila genetics allows for rapid identification and manipulation of genes and/or cellular processes involved in HH signaling.

The term “modulate” as used herein refers to a change or alteration in the biological activity of the Hedgehog signaling pathway, ER stress pathway, Unfolded Protein Response, or a target signalling pathway thereof. In one embodiment the modulator is an “antagonist” or “inhibitor” which blocks, at least to some extent, the normal biological activity or oncogenic activity of the Hedgehog signalling pathway. Antagonists and inhibitors may include proteins, nucleic acids and may include antibodies or small molecules. In another embodiment the modulator is an agonist or “activator” of the Hedgehog signalling pathway, ER stress pathway, Unfolded Protein Response, or a target signalling pathway thereof.

The term “Smoothened (SMO)” as used herein refers to a member of the G-protein coupled receptor (GPCR) superfamily, which functions as the requisite signal transducing molecule of the Hedgehog (Hh) pathway. Smoothened is a G protein-coupled receptor [7] protein encoded by the SMO gene of the hedgehog pathway conserved from flies to humans. It is the molecular target of the teratogen cyclopamine [8]. Cellular localization plays an essential role in the function of SMO. Binding of the Patched receptor by the sonic hedgehog ligand leads to translocation of SMO to the primary cilium. Furthermore, SMO that is mutated in the domain required for ciliary localisation cannot contribute to pathway activation [9]. SMO has also been shown to bind the kinesin motor protein Costal-2 to regulate localization of the Ci (Cubitus interruptus transcription factor) complex in Drosophila [10]. In mammalian systems, SMO associates with the Cos2 ortholog, Kif7, to regulate SMO ciliary localization [11]. SMO can function as an oncogene. Activating SMO mutations can lead to unregulated activation of the hedgehog pathway and cancer [12].

As used herein, the term “primary cilium” is used throughout the specification to describe a sensory organell that projects from the cell basal body and is involved in reception of signals from surrounding cells/extracellular environment.

As used herein, the term “NIH3T3 cells” is used throughout the specification to describe a specific mouse embryo fibroblast cell line.

As used herein, the terms “NPI-0052” or “salinosporamide A” is used throughout the specification to describe (4R,5S)-4-(2-chloroethyl)-1-((1S)-cyclohex-2-enyl(hydroxy)methyl)-5-methyl-6-oxa-2-azabicyclo[3.2.0]heptane-3,7-dione and is also known as Marizomib.

As used herein, the terms “carfilzomib” or “PR-171” is used throughout the specification to describe (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide.

As used herein, the terms “CEP-18770” or “delanzomib” is used throughout the specification to describe (R)-1-((2S,3R)-3-hydroxy-2-(2-phenylpicolinamido)butanamido)-3-methylbutan-2-ylboronic acid.

As used herein, the terms “tanespimycin” or “KOS-953” or “17-AAG” or “17-Allylamino-17-demethoxygeldanamycin” is used throughout the specification to describe (4E,6Z,8S,9S,10E,12S,13R,14S,16R)-13-hydroxy-8,14-dimethoxy-4,10,12,16-tetramethyl-3,20,22-trioxo-19-(prop-2-en-1-ylamino)-2-azabicyclo[16.3.1]docosa-1(21),4,6,10,18-pentaen-9-yl carbamate.

As used herein, the terms “alvespimycin” or “KOS-1022” or “17-DMAG” is used throughout the specification to describe 17-Demethoxy-17-[[2-(dimethylamino)ethyl]amino]geldanamycin.

As used herein, the terms “retaspimycin” or “IPI-504” is used throughout the specification to describe 18,21-Didehydro-17-demethoxy-18,21-dideoxo-18,21-dihydroxy-17-(2-propenylamino)geldanamycin.

As used herein, the terms “PU-H71” is used throughout the specification to describe 6-Amino-8-[(6-iodo-1,3-benzodioxol-5-yl)thio]-N-(1-methylethyl)-9H-purine-9-propanamine.

As used herein, the terms “SNX-2112” is used throughout the specification to describe 4-(6,6-dimethyl-4-oxo-3-(trifluoromethyl)-4,5,6,7-tetrahydro-1H-indazol-1-yl)-2-(((1r,4r)-4-hydroxycyclohexyl)amino)benzamide.

As used herein, the terms “Eeyarestatin I” or “EerI” is used throughout the specification to describe 3-(4-Chlorophenyl)-4-[[[(4-chlorophenyl)amino]carbonyl]hydroxyamino]-5,5-dimethyl-2-oxo-1-imidazolidineacetic acid 2-[3-(5-nitro-2-furanyl)-2-propen-1-ylidene]hydrazide.

As used herein, the terms “versipelostatin” is used throughout the specification to describe a compound with the structure:

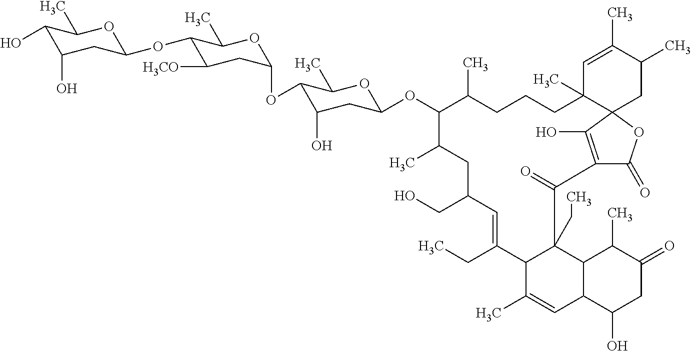 [SMILES: CC/C1=C\C(CO)CC(C)C(OC2CC(O)C(OC3CC(OC)C(OC4CC(O)C(O)C(C)O4)C(C)O3)C(C)O2)C(C)CCCC2(C)C=C(C)C(C)CC23OC(=O)C(=C3O)C(=O)C2(CC)C1C(C)=CC1C(O)CC(=O)C(C)C12]

and related derivatives.

As used herein, the terms “(−)-epigallocatechin gallate” or “EGCG” is used throughout the specification to describe (2R,3R)-5,7-dihydroxy-2-(3,4,5-trihydroxyphenyl)-3,4-dihydro-2H-1-benzopyran-3-yl 3,4,5-trihydroxybenzoate.

As used herein, the terms “epidermal growth factor (EGF)-SubA” is used throughout the specification to describe an engineered fusion protein. An example of this protein is described in Backer, J. M. et al. (2009) [13].

As used herein, the terms “irestatins” is used throughout the specification to describe a series of compounds including irestatin 9389: 2-(3-cyano-4-(trifluoromethyl)-6,7-dihydro-5H-cyclopenta[b]pyridin-2-ylthio)-N-(4-methylthiazol-2-yl)acetamide.

As used herein, the terms “g-202” is used throughout the specification to describe a thapsigargin derived pro-drug that targets the enzyme PSMA.

As used herein, the terms “delta(9)-tetrahydrocannabinol (THC)” is used throughout the specification to describe (−)-(6aR,10aR)-6,6,9-trimethyl-3-pentyl-6a,7,8,10a-tetrahydro-6H-benzo[c]chromen-1-ol.

As used herein, the term “salts,” as used herein, refers to any salt that complexes with identified compounds contained herein while retaining a desired function, e.g., biological activity. Examples of such salts include, but are not limited to, acid addition salts formed with inorganic acids (e.g. hydrochloric acid, hydrobromic acid, sulfuric acid, phosphoric acid, nitric acid, and the like), and salts formed with organic acids such as, but not limited to, acetic acid, oxalic acid, tartaric acid, succinic acid, malic acid, fumaric acid, maleic acid, ascorbic acid, benzoic acid, tannic acid, pamoic acid, alginic acid, polyglutamic, acid, naphthalene sulfonic acid, naphthalene disulfonic acid, and polygalacturonic acid. Pharmaceutically acceptable salts also include base addition salts, which may be formed when acidic protons present are capable of reacting with inorganic or organic bases. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the invention. Unless otherwise specifically stated, the present invention contemplates pharmaceutically acceptable salts of the considered pro-drugs.

As used herein, the term “effective,” as that term is used in the specification and/or claims, means adequate to accomplish a desired, or hoped for result.

As used herein, “room temperature” or “RT” refers to approximately 22° C.

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention. The bulk of the work was performed in the Drosophila system, a robust model of both physiological and pathphysiological Hedgehog signaling.

FIG. 1 shows that active Smo mutants demonstrate attenuated activity at a temperature that induces ER stress. (A) Growth at 29° C. induces an ER stress response. The ER stress sensor UAS-Xbp1-GFP was expressed in salivary glands under control of sgs3-Gal4. Crosses were performed at 22° C. or 29° C., as indicated. Individual salivary glands are outlined in white. Under conditions of low ER stress, Xbp1is not in frame with GFP, and minimal GFP expression is observed (A′). Upon ER stress induction, Xbp1 is alternately spliced to place it in frame with GFP, resulting in a robust GFP signal (A). Scale bar represents 50 um. (B-E) Active Smo mutants are temperature sensitive. Wild type, C320A and C339A Myc-Smo proteins were expressed at 22° C. (C-E) or 29° C. (C′-E′) under control of MS1096-Gal4 driver. Wild type Myc-Smo did not induce a phenotype at 22° C., but induced a mild phenotype at 29° C. (C′ compared to C). Conversely, C320A and C339A Smo mutants induced mild phenotypes at 29° C. and strong phenotypes at 22° C. (D and E), suggesting that their activity is reduced under conditions of thermal stress. MS1096 driver wing serves as control (B). For all conditions representative salivary glands or wings are shown.

FIG. 2 shows active Smo mutants are temperature sensitive in vitro. (A). C18 cells were transfected with empty vector, wild type, C320A or C339A Myc-Smo expression vectors (pAcmyc-smo) in the presence of pAc-hh or empty vector, and control or smo 5′UTR dsRNA, as indicated. Reporter gene activity was determined from cells cultured at 22° C. or 29° C., as indicated. Whereas wild type Myc-Smo rescued ptcΔ136-luciferase activity at both temperatures, C320A and C339A were compromised in their ability to modulate reporter gene activity at the restrictive 29° C. temperature. The control Hh response at each temperature was set to 100%. Reporter gene activity is shown as percent activity relative to the control Hh response. Hh reporter gene activity was normalized against a pAc-renilla control. Error bars indicate s.e.m. (B). C18 cells were transfected with hh, wild type myc-smo or mutant myc-smo expression vectors, as indicated. The Hh response for each temperature was set to 100%. Reporter activity induced by the indicated Myc-Smo protein in the wild type smo background is shown relative to the Hh response. Hh reporter gene activity was normalized against a pAcrenilla control. Error bars indicate s.e.m.

FIG. 3 shows active Smo mutants are largely retained in ER. Wild type (A), C320A (B) and C339A (C) Myc-Smo proteins were expressed in S2 cells at permissive (A-C) or restrictive (A′-C′) temperatures. Myc-Smo was visualized by indirect immunofluorescence. Smo (Myc) is red, Phalloidin (PM marker) is blue and Cal-GFP-KDEL (ER marker) is green. Wild type Smo was vesicular at both temperatures (A-A′). Mutants overlapped with the ER marker at both temperatures (B-C). Scale bar represents 5 um. (D). Activating Smo mutants are not post-ER glycosylated. Whole cell lysates from C18 cells expressing Hh and wild type, C320A or C339A Myc-Smo proteins at 22° C. were treated with the deglycosylating enzyme EndoH, as indicated. Post-ER glycosylated, EndoH-resistant forms of phospho-Smo could be detected for the wild type protein. All of the C320A and C339A Smo protein was EndoH-sensitive, indicative of them being retained in the ER. Kinesin serves as loading control.

FIG. 4 shows active Smo mutants are destabilized at the restrictive temperature. pAc-mycsmo vectors encoding wild type (A, D), C320A (B, E) and C339A (C, F) Myc-Smo proteins were co-transfected into S2 cells with pAc-GFP at permissive (22° C.) and restrictive (29° C.) temperatures. Cells were stained for Smo by indirect immunofluorescence using anti-Myc (magenta) and imaged by confocal microscopy. Whereas wild type Myc-Smo (A-A′) and GFP (D-F and D′-F′) stability and expression were not significantly affected by temperature, both active mutants were destabilized at the restrictive temperature (B-C compared to B′-C′). Multiple fields of cells were examined over two independent experiments. Representative fields are shown. Scale bar represents 50 um. (G) Smo protein is destabilized at the restrictive temperature. Western blot analysis of whole cell lysates from C18 cells expressing wild type or C339A Myc-Smo proteins revealed C339A protein levels to be decreased at 29° C. Wild type Smo was not destabilized at 29° C. Kinesin (Kin) is the loading control.

FIG. 5 shows murine (mouse) Smo mutants are ER localized and temperature sensitive. (A-A′) Murine Smo (mSmo) mutants are not post-ER glycosylated. Lysates prepared from NIH3T3 cells expressing wild type, C299A and C318A mSmo proteins were treated with vehicle (−), EndoH, or additional deglycosylating agents PNGase, O-glycosidase and/or the dephosphorylating enzyme lambda-phosphatase as indicated (+). Samples were analyzed by western blot. The post-ER glycosylated form, present only with wild type mSmo, was not affected by EndoH (post-ER, A-A′), but was affected by PNGase and O-glycosidase (deglycosylated, A-A′). The ER localized forms of wild type and each of the active mutants were sensitive to EndoH, demonstrating a faster mobility after treatment (deglycosylated, A′). EndoH and PNGase treated C299A and C318A have identical mobilities, suggesting that they lack post-ER glycosylation (deglycosylated, A). Lambda-phosphatase does not affect mobility, indicating that wild type mSmo is not phosphorylated in the absence of Shh (A′). Tubulin is the loading control. (B) Oncogenic mSmoM2 is largely ER-retained. Lysates from NIH3T3 cells expressing wild type or M2 mSmo proteins were treated with deglycosylating agents as in (A). A significant pool of EndoH-resistant post-ER protein was evident for wild type mSmo. The bulk of mSmoM2 was EndoH-sensitive. The post-ER pool of mSmoM2 was modest, but detectable (lane 6, post ER label). Tubulin is the loading control. (B′) Indirect immunofluorescence of mSmoM2 in NIH3T3 cells demonstrates that whereas a pool of mSmoM2 (green) is detected in the primary cilium (ciliary slice, arrow), the bulk of the protein co-localized with the ER-resident protein GRP94 (red, ER slice). DAPI (blue) marks the nucleus. Scale bar represents 20 um. (C-C′) Active mSmo mutants are temperature sensitive. (C). NIH3T3 cells were grown at 37° C. for ˜44 hours, then maintained at 37° C. or shifted to 40° C. for an additional 4 hours prior to lysis, as indicated. Induction of the ER stress sensor CHOP was assessed by western blot of whole cell lysates. Tubulin serves as loading control. (C′). NIH3T3 cells expressing wild type, C318A or M2 mSmo proteins were cultured as in (C). Whereas ER (white arrowhead) and post-ER (black arrowhead) forms of wild type Smo were not significantly affected by temperature shift, both of the mutants were destabilized at the high temperature. Destabilization of the ER-resident forms of wild type and both mutants at 40° C. was attenuated by treating cells with the proteasome inhibitor MG132, suggesting that they are cleared by ERAD. The post-ER form of wild type Smo was unaffected by MG132 treatment. Tubulin is the loading control. (D). ERAD attenuation stabilizes mutant mSmo proteins. NIH3T3 cells expressing wild type, C318A or M2 mSmo proteins were transfected with control or Hrd1 siRNA as indicated. Cells were shifted to 40° C. for 4 hours prior to lysis. Hrd1 knockdown stabilizes ER-retained mutant mSmo proteins, but does not affect the post-ER form of wild type mSmo (lanes 1-6 compared to 7-12). Tubulin is loading control.

FIG. 6 shows that the UPR-inducing compound thapsigargin attenuates oncogenic Smo signaling. (A). Active mSmo mutants are destabilized by thapsigargin NIH3T3 cells expressing wild type, C318A or M2 mSmo proteins were treated with 1 uM thapsigargin (Thaps, +) or vehicle control (−), as indicated for 4 hours prior to lysis. Western blot of whole-cell lysates revealed C318A and M2 smo proteins to be destabilized in response to drug treatment. Wild type mSmo was not significantly affected. Tubulin is the loading control. (B). Thapsigargin attenuates mSmoM2-induced pathway activity. RNA was harvested from NIH3T3 cells expressing either wild type or M2 mSmo proteins. qPCR analysis revealed that over-night treatment with 200 nM thapsigargin results in specific attenuation of mSmoM2-induced gli1 expression. Expression is shown as fold induction over the wild type mSmo vehicle control. Expression is normalized to the GAPDH reference gene.

FIG. 7 shows RNA was harvested from NIH3T3 cells expressing either wild type or M2 mSmo proteins. qPCR analysis revealed that thapsigargin treatment results in a modest increase in smo expression. Expression is shown as fold induction over the wild type mSmo vehicle control. Expression is normalized to the GAPDH reference gene.

FIG. 8 shows that thapsigargin ameliorates ectopic signaling by an active Smo mutant in vivo. (A) Transgene expression is unaffected by thapsigargin. MS1096>GFP larvae were grown on media containing vehicle or thapsigargin, as indicated. Wing imaginal discs from 3rd instar larvae from both conditions demonstrated comparable GFP expression (green). DAPI (magenta) marks the nuclei. (B-D) Thapsigargin prevents Myc-SmoC320A-induced Hh gain-offunction wing phenotypes. Larvae expressing wild type or C320A UAS-myc-smo under control of MS 1096-Gal4 were grown at 22° C. on food containing vehicle (C-D) or thapsigargin (C′-D′). Representative wings from adult flies are shown. MS1096-Gal4 driver wing serves as control (B). Thapsigargin did not affect wings expressing wild type Smo (C-C′). The C320A-induced phenotype was significantly attenuated by drug, allowing for development of near-normal adult wings (D′ compared to D). (E-F) SmoC320A-induced downstream pathway activity is ameliorated by thapsigargin. Wing imaginal discs from SmoC320A-expressing larvae grown on vehicle- (E) or thapsigargin- (F) containing food at 22° C. were stained for Myc-SmoC320A (Myc, green) and full-length Ci (red). Note the significantly reduced SmoC320A protein level and reduced Ci stabilization in thapsigargin-treated discs (F compared to E). For all wing disc images, discs are shown with dorsal up and anterior left. Scale bar represents 100 um.

### DETAILED DESCRIPTON OF THE INVENTION

### I. Introduction

The invention relates to methods for the treatment of cancer with SMO mutations as well as diagnosing tumors with SMO mutations. The Hedgehog (Hh) signaling pathway provides essential patterning information during development, and is frequently activated in cancer. Inappropriate Hh signaling is causative in medulloblastoma and basal cell carcinoma, and has been implicated in cancers of the lung, breast, prostate and digestive tract. Smoothened (SMO), a member of the G-protein coupled receptor (GPCR) superfamily, functions as the requisite signal transducing molecule of the Hedgehog (Hh) pathway. Activating mutation of Smo is one mechanism by which the Hh pathway can become inappropriately activated in cancer; a number of oncogenic Smo mutations have been identified in sporadic basal cell carcinomas and medulloblastomas. In humans, Shh signaling has been implicated in breast, lung cancer, pancreatic cancer and cancers of the digestive tract. However, in these cancers, it is not believed to be causative. Shh signaling may provide a growth advantage or play a survival role once the tumor has formed. Cancers where Shh signaling is known to be causative include basal cell carcinoma, medulloblastoma and rhabdomyosarcoma. The present invention relates to treating tumors having SMO mutations as well as diagnosing tumors with SMO mutations. The present invention describes that ER stress-inducing or UPR modulating compounds might fill a clinical need for additional methods of targeting the Hh pathway, either in frontline combination therapy or in salvage therapy for relapsed patients who develop resistance to the available SMO inhibitor and offer a significant advantage over SMO-specific small molecules. Because ER stress modulators exploit a cellular process that is distinct from the Hh signaling pathway, their efficacy should be unaltered by acquired SMO mutation.

The an illustrative example of on aspect of the invention includes experiments with the UPR-inducing compound thapsigarin. Thapsigargin is a compound is derived from a plant (Thapsia garganica) and is a sesquiterpene lactone, tumorigenic in mammalian cells and possibly an antiparasitic agent. Mechanistically, it causes the endoplasmic reticulum to dump its calcium stores into the extracellular space. Cells try to replenish those stores by pumping calcium into the cytoplasm through calcium channels in the plasma membrane.

One reference, Xie, J. et al. (1998) Activating Smoothened Mutations in Sporadic Basal-Cell Carcinoma, Nature 391(6662), 90-92 [12] describes various activating SMO mutations in actual cancer patients with basal-cell carcinomas. The SMO mutations were found only in the tumor DNA and not the patient's normal cell DNA. The authors conclude that their results implicate the Hedgehog signaling pathway in tumorigenesis, especially in the skin BCCs consistently show activation of the Hedgehog signaling pathway, as judged by increased PTCH mRNA. They further suggest that they have provided evidence that this activation can be caused by missense mutations in SMO, supporting a model in which SMO activity drives the Hedgehog signaling pathway, the tumor suppressor PTCH represses signaling by SMO, and SHH relieves this repression. Therefore SMO can be classified as a proto-oncogene. The authors clearly state that “pharmacological inhibition of SMO or of downstream effectors of this pathway could provide an effective treatment for BCCs and perhaps for other cancers as well.” This reference does not disclose specific possible inhibitors of SMO or use of an ER Stressor compound to treat cancers with SMO mutations

One reference, Ng, J. M. Y. and Curran, T. (2011) The Hedgehog's Tale: Developing Strategies for Targeting Cancer, Nat. Rev. Cancer 11(7), 493-501 [14] discloses that cyclopamine and GDC-0449 can inhibit SMO function and completely block all HH pathway signaling regardless of ligand. The reference also discloses that mutation of SMO is associated with several cancer types. This reference also suggests simply inhibiting SMO may not actually inhibit SHH related cancers due to various mutations in SMO. The authors cite the lack of appropriate biomarkers makes it challenging to develop robust criteria for the stratification of patients with tumors other than BCC or medulloblastoma for treatment with SMO inhibitors. This reference does not disclose use of an ER Stressor compound to treat cancers with SMO mutations.

One reference, Dijkgraaf, G J. P. et al. (2011) Small Molecule Inhibition of GDC-0449 Refractory Smoothened Mutants and Downstream Mechanisms of Drug Resistance, Cancer Res. 71(2), 435-444 [15] discloses that several functional mutations of SMO can resist the SMO targeting drug GDC-0449, a problem found in cancer relapse mutations. This highlights the need for a treatment that can address cancers that have appeared after previous therapy and are resistant to current therapeutics. The authors screened various hedgehog pathway inhibitors for those that would have antagonist activity against these SMO mutations. They identified bis-amide compound N-(4-chloro-3-(3-chlorobenzamido)phenyl)-6-((3S,5R)-3,5-dimethylpiperazin-1-yl)nicotinamide, shown to the right, as the most promising prospect. However, compound 5 does not appear to stress the ER or induce the UPR. This reference does not disclose the use of an ER Stressor compound to treat cancers with SMO mutations or treat those that have become resistant to other HH pathway inhibitors.

One reference, Li, X. et al. (2011) Unfolded Protein Response in Cancer: The Physician's Perspective, J. Hematol. Oncol. J Hematol Oncol 4(1), 8-17 [16] discloses a myeloma cell study demonstrated that HSP90 inhibitors, 17AAG (17-allylamino-17-demethoxygeldanamycin) and radicicol, similar to tunicamycin (TM) and thapsigargin (TG) (known unfolded protein response (UPR) activators), are capable of activating all three branches of the UPR. The goal of the inhibitors is to ultimately induce a generalized ER response in tumor cells, leading to apoptosis. The references describes multiple examples of UPR-targeted cancer drugs in development: NPI-0052 (salinosporamide A), Carfilzomib (PR-171), PS-341, CEP-18770, Tanespimycin (17-AAG, (17-Allylamino-17-demethoxygeldanamycin), KOS-953), Alvespimycin (KOS-1022, 17-DMAG), Retaspimycin (IPI-504), PU-H71, SNX-2112, Eeyarestatin I (EerI), Versipelostatin, (−)-epigallocatechin gallate (EGCG), Epidermal growth factor (EGF)-SubA, Irestatins, and Delta(9)-Tetrahydrocannabinol (THC). This reference does not disclose the use of ER Stressor compounds to treat cancers with SMO mutations or treat those that have become resistant to other HH pathway inhibitors.

One reference, Olive, K. P. and Tuveson, D. “Hedgehog Pathway Inhibitors,” United States Patent Application 20120020876 application Ser. No. 13/144992, filed Jan. 22, 2010. (Published Jan. 26, 2012) [17] discloses methods of treating or preventing tumor metastasis with a hedgehog pathway inhibitor. “In certain embodiments, the tissue comprises autochthonous tissue, stromal tissue, ischemic tissue, or tumor tissue. In certain embodiments the tumor tissue exhibits Hedgehog pathway activation. In certain embodiments, the Hedgehog pathway activation is characterized by one or more of phenotypes selected from group consisting of a Patched (Ptc) loss-of-function phenotype or a Smoothened (Smo) gain-of-function phenotype.” This reference does not describe SMO mutations, only phenotypes and describes administration of a hedgehog pathway inhibitor and an agent. This reference does not disclose the use of an ER Stressor compound to treat cancers with SMO mutations or treat those that have become resistant to other HH pathway inhibitors.

One reference, Yauch, R. L. et al. (2009) Smoothened Mutation Confers Resistance to a Hedgehog Pathway Inhibitor in Medulloblastoma, Science 326(5952), 572-574 [18] discloses that mutated SMO can infer resistance to therapeutics designed to target SMO, namely GDC-0449. That mutations can result in resistance leads to the authors to conclude that this highlights the need to either identify second-generation SMO inhibitors capable of overcoming acquired resistance, identify inhibitors targeting downstream signaling molecules, or potentially initiate earlier treatment before therapy with radiation or other DNA-damaging agents. This reference does not disclose the use of an ER Stressor compound to treat cancers with SMO mutations or treat those that have become resistant to other HH pathway inhibitors.

The Hedgehog signal transduction pathway, which is essential for pattern formation during development, is implicated as playing causative and survival roles in a range of human cancers. Accordingly, the requisite signal transducing component of the pathway, Smoothened, has revealed itself to be an efficacious therapeutic target. Despite clinical success, challenges remain in cases where oncogenic Hedgehog signaling is induced by somatic Smoothened mutation, and also in cases where tumors become resistant to Smoothened-specific antagonists. Herein, it is shown that Hedgehog pathway activity driven by active Smoothened mutants, including oncogenic Smoothened M2, is specifically attenuated by ER stressors that activate the unfolded protein response (UPR). Further, herein it is demonstrated that the UPR-inducing compound thapsigargin effectively eliminates phenotypes induced by Smoothened gain-of-function mutants in transgenic Drosophila, suggesting that alteration of ER homeostasis may be a viable method of targeting Hedgehog signaling in disease. The Drosophila model system offers a powerful tool to dissect the HH signaling cascade because HH pathway components are tightly conserved from Drosophila to human, HH phenotypes are well characterized in Drosophila, and the power of Drosophila genetics allows for rapid identification and manipulation of genes and/or cellular processes involved in HH signaling. Given that a number of ER stress and UPR-modulating compounds are currently being evaluated for clinical use, it is proposed that manipulation of the UPR may provide an immediate strategy for targeting oncogenic Smoothened signaling in cancer.

The Hedgehog (Hh) signaling pathway provides essential patterning information during development, and is frequently activated in cancer (Barakat et al., 2011 [19]; Ingham & McMahon, 2001 [20]; Jiang & Hui, 2008 [21]). While not limiting the present invention to any particular theory or mechanism, inappropriate Hh signaling is causative in medulloblastoma, basal cell carcinoma, and rhapbomyosarcoma, and has been implicated in a number of additional cancers including those of the lung, breast, prostate and digestive tract (Barakat et al., 2011 [19]; Berman et al., 2003 [22]; Fan et al., 1997[23]; Goodrich et al., 1997 [24]; Karhadkar et al., 2004[25]; Watkins et al., 2003 [26]; Xie et al., 1998 [12]; Yuan et al., 2007 [27]). While not limiting the present invention to any particular theory or mechanism, smoothened (Smo), a member of the G-protein coupled receptor superfamily, functions as the requisite signal transducing molecule of the Hedgehog (Hh) pathway (Alcedo et al., 1996 [28]; van den Heuvel & Ingham, 1996 [29]). Accordingly, oncogenic mutation of Smo is one mechanism by which the Hh pathway can become inappropriately activated in cancer (Lain et al., 1999 [30]; Xie et al., 1998 [12] and www.sanger.ac.uk).

A set of active Sino mutants were recently described that, like oncogenic Smo, induce ligand independent Hh pathway activity (Carroll et al., 2012 [31]). These mutants, C320A and C339A in the Drosophila protein and C299A and C318A in the murine protein, are predicted to break disulfide bonds that stabilize a regulated conformation of the Smo extracellular loop domain (Cook et al., 1996 [32]; Karnik et al., 1988 [33]; Moro et al., 1999 [34]). Consistent with the prediction that alteration of such bonds would result in a misfolded protein, all of these mutants are largely retained in the ER (Carroll et al., 2012 [31]). Similarly, the oncogenic Smo mutant, SmoM2, has been reported to be largely ER-localized (Chen et al., 2002 [35]; Incardona et al., 2002 [36]). However, a small pool of M2 escapes the ER, and traffics to the primary cilium through an atypical Rab 8-dependent secretory route (Hoffmeister et al., 2011 [37]; Rohatgi et al., 2009 [38]; Wong et al., 2009 [39]). This transport from the ER to the primary cilium is important for M2 oncogenic activity, as genetic ablation of the primary cilium attenuates M2-induced tumor formation in mice (Han et al., 2008 [40]; Wong et al., 2009 [39]).

Accumulation of misfolded protein in the ER adversely affects ER homeostasis (Walter & Ron, 2011 [4]). This can result in high ER stress, leading to induction of the unfolded protein response (UPR), a compensatory process aimed at ameliorating ER stress and preventing stress-induced cell death (Hetz, 2012 [41]; Walter & Ron, 2011 [4]). The UPR is organized into three branches, each controlled by a unique upstream activator. The PERK branch triggers phosphorylation of elongation factor 2α to attenuate translation of nascent proteins bound for the ER (Harding et al., 1999 [1]). The ATF6 and IRE1α branches activate transcription factors that drive expression of UPR target genes involved in protein quality control and ER associated degradation (ERAD), a process that targets misfolded proteins for retro-translocation from the ER to the cytoplasm where they undergo proteasome-mediated degradation (McCracken & Brodsky, 1996 [2]; Tirasophon et al., 2000 [3]; Walter & Ron, 2011 [4]; Yoshida et al., 1998 [5]). Although it is not necessary to understand the mechanism of an invention, it is believed that persistent ER stress that cannot be corrected by the UPR will eventually result in apoptosis (Walter & Ron, 2011 [4]). However, the exact mechanisms by which the ER stress signals for induction of apoptosis under such conditions are not yet clear. However, while not limiting the current invention, a preferred embodiment of the current invention involves engaging the UPR to drive degradation of the active Smo mutants. Because the cells are “addicted” to Hh pathway activity driven by the oncogenic Smo mutant, such cells will likely die or be susceptible to another chemotherapeutic. This mechanism is not expected to induce apoptosis through an ER signal. In fact, in assays, increased apoptosis in response to SmoM2 destabilization by the UPR could not be detected.

Given its ability to influence cellular homeostasis and apoptosis, it is no surprise that the UPR has become an attractive target for therapeutic intervention in cancer. Because tumor cells typically exist in nutrient-poor, hypoxic conditions that readily induce ER stress, it has been widely acknowledged that therapeutic manipulation of the UPR under such conditions may serve as an Achilles' heel for targeting tumor cells (Li et al., 2011 [16]; Liu & Ye, 2011 [42]). Accordingly, a number of small molecule ER stress modulators, both UPR agonists and antagonists, are currently in or en route to the clinic (Li et al., 2011 [16]).

The increased localization of active Smo mutants to the ER prompted a test as to whether Smo mutants might be sensitive to alteration of ER homeostasis and induction of the UPR. Herein are findings demonstrating that active Smo mutants, including extracellular loop C to A mutants and the oncogenic mutant SmoM2, are specifically destabilized by the UPR under conditions of thermally- and chemically-induced ER stress. Under these conditions, signaling by active Smo mutants is attenuated by their selective degradation via ERAD. Consistent with these results, the ER stress and UPR inducing compound thapsigargin blocks Smo-mediated Hh gain-of-function phenotypes in vivo in Drosophila. These findings suggest that ER stress modulators that trigger the UPR may represent a novel therapeutic window to be evaluated for treatment of Hh-dependent cancers. Such compounds may be particularly efficacious in cancers initiated by oncogenic Smo and/or in tumors harboring Smo mutations that demonstrate reduced sensitivity to the current cache of Smo inhibitors (Rudin et al., 2009 [43]; Taipale et al., 2000 [8]; Yauch et al., 2009 [18]).

### II. Results

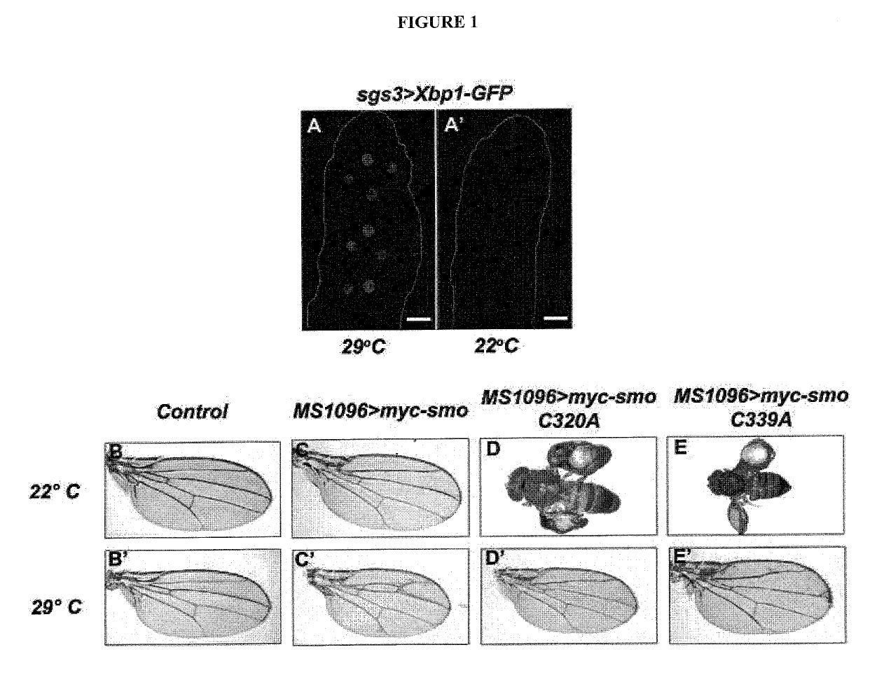

To determine whether activity of the ER-retained active Drosophila Smo mutants C320A and C339A would be affected by induction of a cellular stress response, thermal stress was induced by performing crosses at high temperature (29° C.). To determine whether the UPR would be induced at this temperature, ER stress was monitored by expression of an Xbp1-GFP stress sensor (Ryoo et al., 2007 [44]). GFP expression is activated by an ER stress-stimulated, IRE1α-mediated alternate splice reaction that places GFP in frame with the Xbp1 transcript (Iwawaki et al., 2004 [45]; Ryoo et al., 2007 [44]). Whereas minimal expression of GFP was observed in salivary glands of reporter flies at 22° C., strong induction of GFP was observed at 29° C. (FIGS. 1A-A′), confirming induction of an ER stress response at the high temperature.

It has been previously demonstrated that when expressed under control of the UAS/GAL4 system at 25° C., wild type Smo transgenes induce a modest Hh gain-of-function phenotype and SmoC320A and SmoC339A transgenes induce strong phenotypes (Carroll et al., 2012 [31]). When these same transgenes were expressed at 22° or 29° C., wild type Myc-Smo did not trigger a Hh gain-of-function phenotype at 22° C., but did induce mild ectopic vein formation when expressed at 29° C., likely due to higher-level UAS/GAL4 transgene expression at 29° C. (FIG. 1C compared to C′ and B and Duffy, 2002 [46]). Conversely, expression of the activating C320A and C339A Myc-Smo mutants induced mild phenotypes at 29° C., and dramatic phenotypes including dorsal wing over-growth and wing blistering at 22° C. (FIGS. 1D-E compared to D′-E′). Additionally, whereas both C320A and C339A Myc-Smo transgenic flies enclosed in normal ratios at 29° C., expression of the more active Myc-SmoC339A mutant induced a high degree of pupal lethality at 22° C. Taken together, these results suggest that active Smo mutants induce robust signaling at low temperature, but that their activity is significantly attenuated at a temperature that induces stress response pathways including the UPR (FIG. 1A).

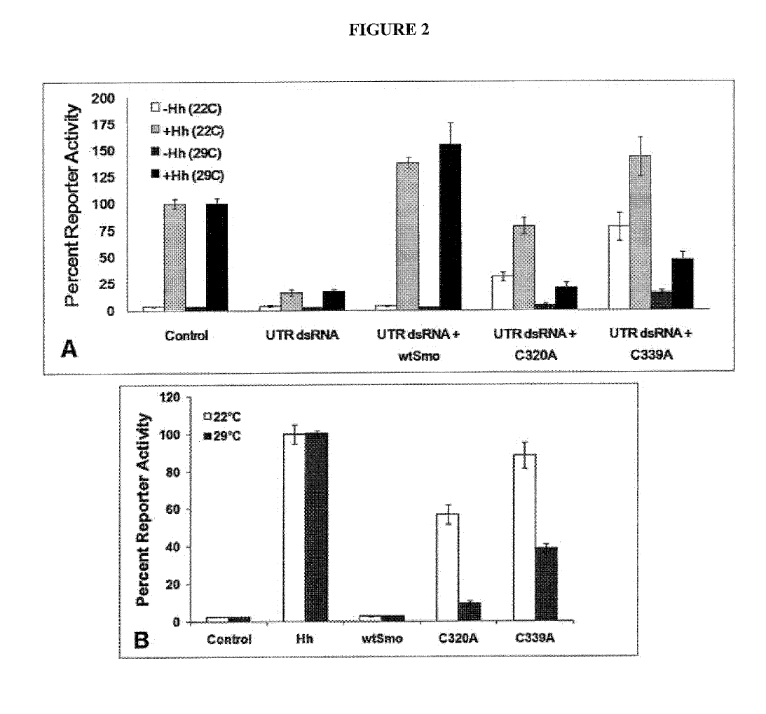

To determine whether the observed in vivo temperature sensitivity was a specific functional effect on mutant Smo proteins, or simply an artifact of in vivo transgene expression, the experiment was switched to an in vitro Clone 8 (C18) cell culture system that allowed for the performance of functional assays at permissive (22° C.) and restrictive (29° C.) temperatures. C18 cells are derived from wing imaginal disc tissue, and possess an intact Hh pathway, making them an ideal cell line to perform biochemical and functional analyses for Hh pathway activity (Aza-Blanc et al., 1997 [47]; Chen et al., 1999 [48]). The ability of wild type and mutant Smo proteins to rescue reporter gene expression was assessed in C18 cells in a dsRNA-mediated smo knockdown background at 22° C. or 29° C. (FIG. 2A). Knockdown of endogenous smo using 5′UTR dsRNA attenuated Hh induced reporter gene expression at both permissive and restrictive temperatures (FIG. 2A, UTR dsRNA). Re-expression of wild type Myc-Smo using cDNA lacking UTR sequence did not alter baseline signaling activity, but rescued Hh-dependent reporter gene induction to similar levels at both temperatures (UTR dsRNA+wtSmo), suggesting that, as was observed in flies, wild type Myc-Smo function is not significantly altered by temperature. Consistent with previous studies performed at 25° C. (Carroll et al., 2012 [31]), both C320A and C339A mutant Myc-Smo proteins significantly elevated baseline signaling and partially (C320A) or fully (C339A) rescued Hh-dependent reporter gene activity in the smo knockdown background at 22° C. (FIG. 2A, white and light gray bars). However, both mutants were compromised in their ability to elevate baseline activity and to rescue Hh-induced reporter gene activity at the restrictive 29° C. temperature (FIG. 2A, dark gray and black bars).

Next the ability of wild type or mutant Myc-Smo proteins to induce ectopic reporter gene activity at permissive or restrictive temperatures in a wild type smo background was examined. Consistent with Smo being post-translationally regulated (Alcedo et al., 2000 [49]; Denef et al., 2000 [50]; Ingham et al., 2000 [51]), provision of exogenous wild type Myc-Smo did not increase baseline signaling at either temperature (FIG. 2B, wtSmo). Conversely, at 22° C., both mutants increased baseline signaling to a level near to (C320A) or equal to (C339A) the control Hh response (FIG. 2B, white bars). This activity was attenuated at 29° C., suggesting that the presence of endogenous Smo does not correct the temperature sensitivity of the C320A or C339A Myc-Smo mutants (FIG. 2B, gray bars). Taken together with the smo rescue reporter assay, these results suggest that the observed in vivo temperature sensitivity of activating Smo mutants is triggered by a molecular mechanism affecting mutant Smo proteins, rather than by altered transgene expression.

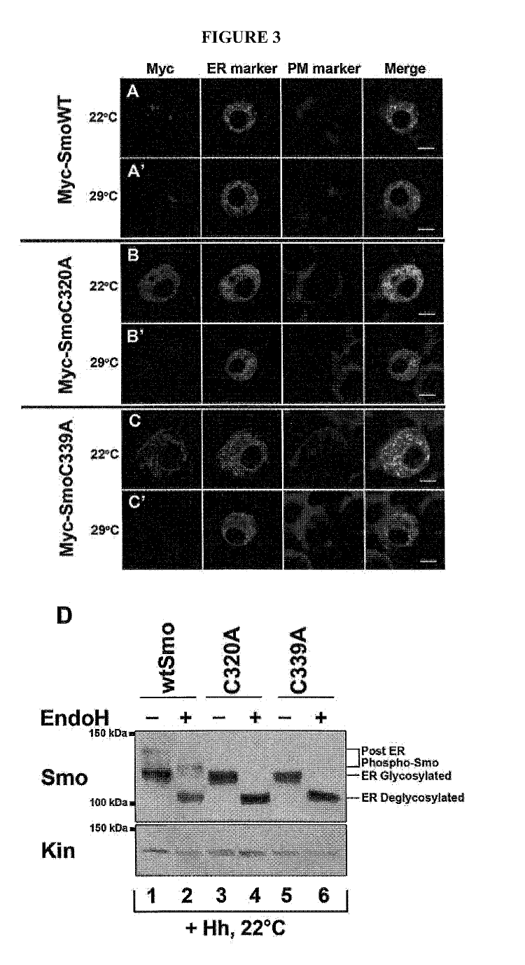

Because high-level Hh pathway activity in Drosophila correlates with accumulation of Smo on the plasma membrane (PM) (Denef et al., 2000 [50]), it was best to determine whether the robust in vitro signaling and strong in vivo phenotypes induced at 22° C. might result from active Smo mutants escaping the ER and localizing to the PM at the permissive temperature. To do so, subcellular localization of wild type and mutant Myc-Smo proteins was examined in Schneider 2 (S2) cells, an embryonic Drosophila cell line commonly used for imaging studies, at 22° C. and 29° C. (FIGS. 3A-C). In the absence of Hh, Smo localizes to intracellular vesicles and recycling endosomes (Incardona et al., 2002 [36]). Accordingly, wild type Myc-Smo localized primarily to punctate structures that did not significantly overlap with the ER marker Calreticulin (Cal)-GFPKDEL (Casso et al., 2005 [52]) at either 22° C. or 29° C. (FIGS. 3A-A′). Myc-SmoC320A and C339A demonstrated a different localization pattern, colocalizing almost completely with the Cal-GFP ER marker at both 22° C. and 29° C. (FIGS. 3B-B′ and C-C′). Obvious colocalization between Myc-SmoC320A or C339A and the PM stain Phalloidin was not detected at either temperature (FIGS. 3B-C), suggesting that the increased signaling activity of these mutants at 22° C. was not the result of a bulk relocalization from the ER to the PM.

To biochemically confirm that the activating mutants were unable to escape the ER, even under conditions that are favorable for high-level Hh signaling, wild type, C320A and C339A Myc-Smo proteins were expressed in C18 cells at 22° C. in the presence of Hh, and processing of their N-linked glycans was assessed. To do so, Endoglycosidase H (EndoH), which cleaves high mannose oligosaccharides that are added in the ER, but does not cleave the more complex oligosaccharides that are processed in post-ER compartments, was utilized. EndoH sensitivity analysis revealed that, whereas wild type Smo was detectable in ER-glycosylated, post-ER glycosylated and Hh-induced phosphorylated forms, C320A and C339A proteins were present only in the EndoH sensitive, ER-resident fraction (FIG. 3D compare lanes 3-6 with 1-2). Taken together, these results support the conclusion that the increased activity observed for C320A and C339A mutants at 22° C. is not due to their relocalization to the PM at the permissive temperature.

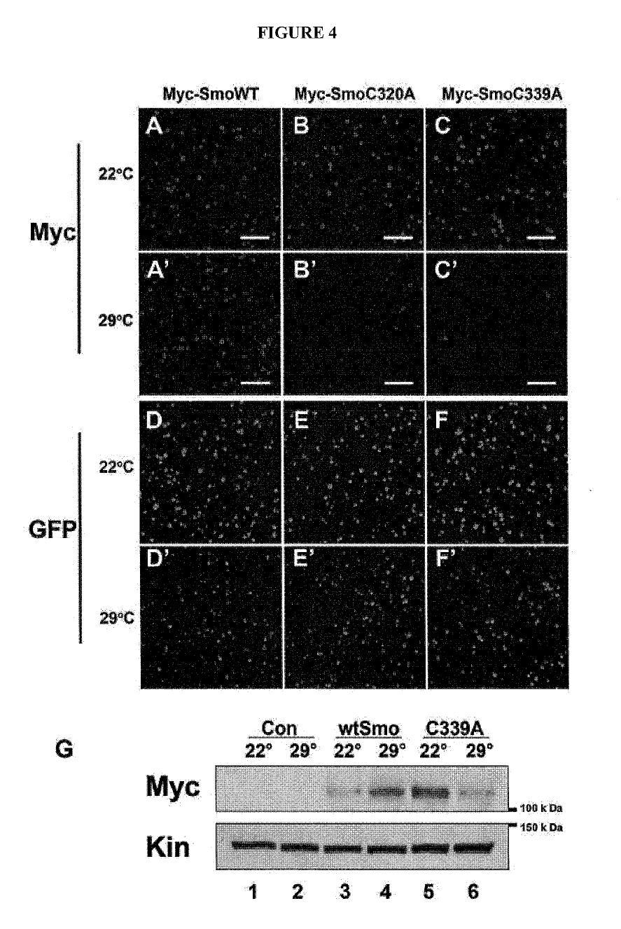

While examining Smo subcellular localization (FIGS. 3A-C), it was noticed that whereas wild type Smo protein stability did not appear to be affected by temperature, C320A and C339A Smo proteins consistently appeared more stable at the permissive 22° C. temperature (FIGS. 3A-C). To determine whether this was a specific stability effect on mutant Smo proteins, and not due to effects on transfection efficiency or in vitro transgene expression at the different temperatures, pAc-GFP was co-transfected with each of the pAc-myc-smo expression vectors into S2 cells at permissive and restrictive temperatures, and examined their expression by gain-equalized immunofluorescence (FIGS. 4A-F). Wild type Myc-Smo and the GFP tracer were not significantly affected by temperature (FIG. 4A compared to A′ and D-F compared to D′-F′). Conversely, Myc-SmoC320A and Myc-SmoC339A were consistently less detectable at the higher temperature (FIGS. 4B-C compared to FIGS. 4B′-C′). To confirm these results biochemically, whole-cell lysates were prepared from C18 cells expressing wild type or the highly active C339A mutant at 22° C. or 29° C., and examined Myc-Smo protein by western blot. Whereas wild type Myc-Smo was not destabilized at the restrictive temperature, the Myc-SmoC339A protein level was significantly reduced (FIG. 4G, lanes 3-4 compared to 5-6). Taken together with the immunofluorescence data, these results suggest that the activating Smo mutants are specifically destabilized at 29° C.

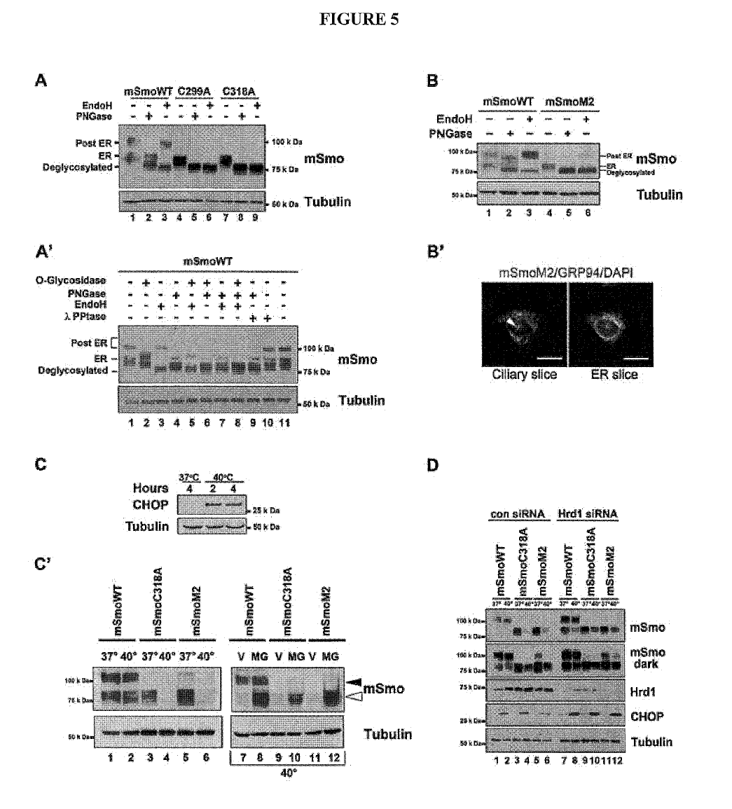

It was previously demonstrated that, like the fly proteins, murine Smo (hereafter referred to as mSmo) mutants corresponding to Drosophila Smo C320A and C339A are also largely ERretained (Carroll et al., 2012 [31]). To biochemically validate ER retention of these mSmo mutants, wild type, mSmoC299A (C320A equivalent) and C318A (C339A equivalent) were expressed in NIH3T3 cells, and examined ER and post-ER glycosylation patterns (FIG. 5A). Whereas wild type mSmo existed in both an EndoH-sensitive ER form and an EndoH-resistant/PNGasesensitive post-ER form, only the ER form of the C299A and C318A active mutants could be detected (FIG. 5A). It was noted that a slow-migrating form of wild type mSmo, even in the presence of PNGase, which cleaves all ER and post-ER N-linked glycosylation moieties (FIG. 5A, lane 2). Lambda phosphatase treatment confirmed that this shift was not due to phosphorylation (FIG. 5A′, lanes 9-10). To determine whether the mobility shift observed for wild type Smo might be the result of a post-ER O-linked glycosylation event, lysates from cells expressing wild type mSmo were treated with O-glycosidase alone and in combination with PNGase and/or EndoH (FIG. 5A′). A modest collapse upon O-glycosidase treatment was observed (FIG. 5A′ lane 2), and complete collapse upon treatment with all three enzymes (FIG. 5A′ lane 8), confirming that the residual shift observed with the wild type protein is likely due to an O-linked glycosylation event.

The murine equivalent of the oncogenic W535L SmoM2 mutant (SmoA1, W539L, hereafter referred to as mSmoM2) induces robust Sonic Hedgehog-independent signaling activity (Taipale et al., 2000 [8]; Xie et al., 1998 [12]). Like C299A and C318A mSmo mutants, a significant fraction of mSmoM2 is retained in the ER (Chen et al., 2002 [35]; Incardona et al., 2002 [36]). In order to examine the ratio of ER-localized to post-ER localized mSmoM2, mSmoM2 was expressed in NIH3T3 cells, and examined ER and post-ER glycosylation status (FIG. 5B). Wild type mSmo demonstrated a near equal distribution between ER and post-ER glycosylated pools (FIG. 5B lanes 1-3). Conversely, the bulk of mSmoM2 protein existed in the EndoH sensitive, ER-localized fraction (FIG. 5B lanes 4-6, Post ER vs. Deglycosylated). Although modest, an EndoH-resistant post-ER form of SmoM2 could be detected (FIG. 5B lane 6), and likely represents mSmoM2 protein that is localized to or is en route to the primary cilium. Accordingly, whereas mSmoM2 could be detected in the primary cilium by indirect immunofluorescence, the bulk of mSmoM2 protein co-localized with the ER resident protein GRP94 (FIG. 5B′ ciliary slice, arrowhead vs ER slice).

Given that the bulk of mSmoC318A and mSmoM2 are ER-localized, it was reasoned that, like the active Drosophila mutants, they would be affected by induction of a thermal ER stress response. Thermal ER stress has been observed in mammalian cells cultured at 40° C. (Xu et al., 2011 [53]). Accordingly, it was observed induction of the ER stress response transcription factor CHOP in NIH3T3 cells incubated at this high temperature for 2-4 hours (FIG. 5C). To assess the effect of thermal ER stress on mSmo proteins, wild type, C318A or mSmoM2 was expressed in NIH3T3 cells cultured at 37° C., then shifted to 40° C. for 4 hours prior to whole-cell lysis (FIG. 5C′). It was found that whereas wild type mSmo was not significantly affected by temperature shift, both C318A and M2 mSmo proteins were significantly destabilized at 40° C. (FIG. 5C′ lanes 3-6 compared to 1-2). This destabilization is the likely result of ER-associated protein degradation (ERAD), as the ER-resident forms of all three Smo variants were stabilized at 40° C. by the proteasome inhibitor MG132 (FIG. 5C′ white arrowhead, lanes 8, 10 and 12 compared to 7, 9 and 11). The post-ER form of wild type Smo was not altered by MG132 treatment (FIG. 5C′ black arrowhead, lanes 7-8).

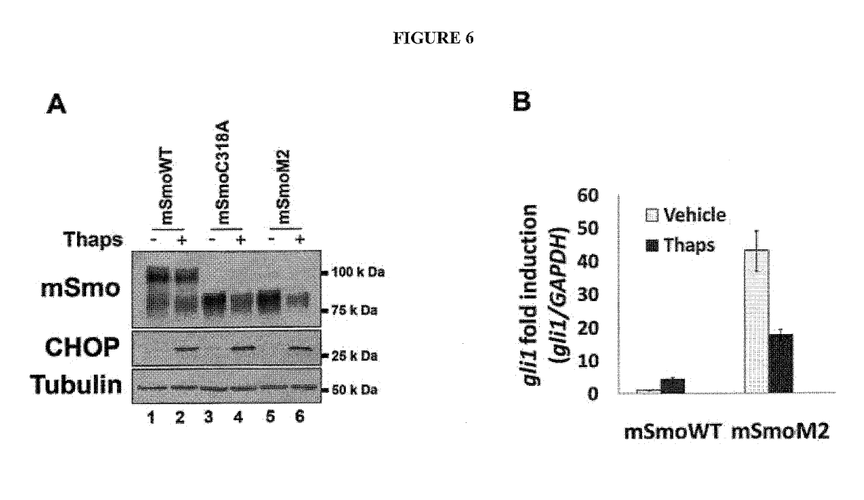

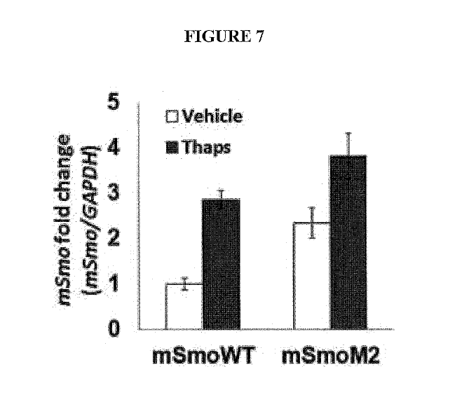

Next, it was decided to determine whether the effects observed following thermal ER stress induction could be recapitulated by an ER stress modulator that is specific to the UPR. To this end, Smo-expressing cells were treated with the potent ER stress inducing compound thapsigargin. Wild type, C318A or M2 mSmo proteins was expressed in NIH3T3 cells, and treated with vehicle or thapsigargin for 4 hours prior to lysis (FIG. 6A). Whereas wild type mSmo stability was not affected by thapsigargin treatment, both C318A and M2 mSmo proteins were destabilized (FIG. 6A lanes 3-6 compared to 1-2), suggesting that in addition to being affected by thermal stress, the active mutants are also sensitive to chemically-induced ER stress. To confirm that thapsigargin-mediated mSmoM2 destabilization was sufficient to attenuate downstream signaling, endogenous gli1 induction was assessed using qPCR (FIG. 6B). mSmoM2-expressing cells demonstrated an approximate 40-fold increase in gli1 expression over that observed in mSmoWT-expressing cells (FIG. 6B white bars). Thapsigargin treatment specifically reduced mSmoM2-mediated gli1 induction approximately 50% to a level ˜18-fold over the mSmoWT baseline. Consistent with this biochemical analyses, thapsigargin did not attenuate gli1 expression in cells expressing wild type mSmo, and instead modestly increased gli1 expression ˜4-fold over baseline (FIG. 6B gray bars). The exact mechanism by which gli1 expression is increased in the wild type mSmo background in response to thapsigargin was not known. However, it was speculated this may be the result of increased transcriptional activity of mSmo expression vectors in the presence of drug; both wild type and M2 transcripts increased modestly in response to drug treatment (FIG. 7).

The in vivo and in vitro results thus far suggest that active Smo mutants that are retained in the ER are sensitive to ER stressors that enhance protein misfolding and induce the UPR, i.e. high temperature and thapsigargin (Li et al., 2000 [54]; Xu et al., 2011 [53]). Wild type Smo is not significantly targeted by the UPR following either thermal or chemical ER stress induction, suggesting that small molecule-mediated UPR modulation may represent a selective process by which to target active Smo mutants in disease. To test this hypothesis, whether thapsigargin could alter mutant Smo-induced Drosophila wing phenotypes was examined. Due to the high level of pupal lethality induced by the Myc-SmoC339A mutant at 22° C., the Myc-SmoC320A mutant was chosen for this assay.

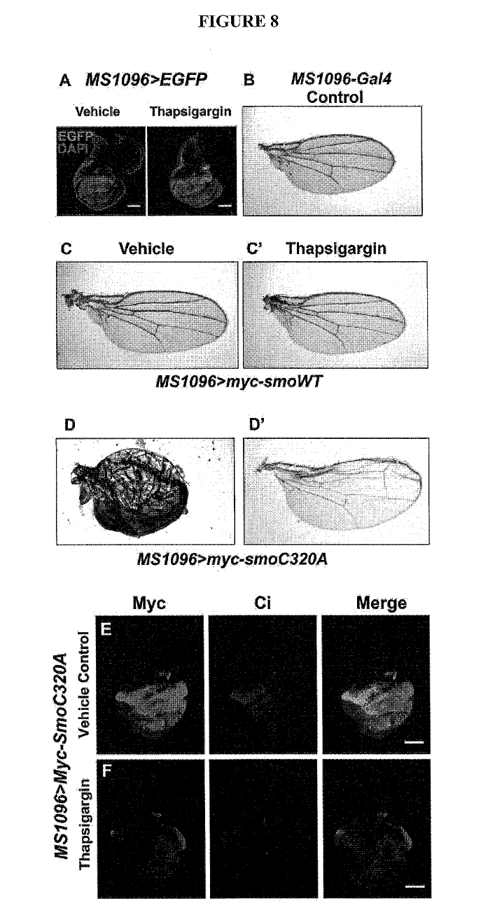

To confirm that thapsigargin treatment of transgenic Drosophila would not nonspecifically alter UAS/GAL4 transgene expression, the effect of feeding thapsigargin or vehicle control to larvae expressing a GFP transgene under control of the MS1096-Gal4 wing pouch driver was first tested (FIG. 8A). GFP expression was unaffected by thapsigargin treatment at 22° C., confirming that the drug does not non-specifically alter transgene expression in vivo. Next either wild type or C320A Myc-Smo proteins were expressed at 22° C. under control of MS1096-Gal4, and grew larvae on food containing either vehicle or thapsigargin (FIGS. 8C-D). Consistent with the in vitro results, wings from wild type Myc-Smo expressing flies were unaffected by thapsigargin treatment (FIGS. 8C-C′ compared to B, control). Conversely, Myc-SmoC320A-expressing thapsigargin-fed larvae demonstrated a significantly reduced Hh gain-offunction phenotype compared to that of the vehicle-fed control (FIG. 8D compared to D′). Consistent with the in vitro data, thapsigargin-mediated induction of the UPR resulted in a significant reduction of Myc-SmoC320A protein in wing imaginal discs dissected from drug-fed larvae (FIG. 8F compared to E, green). This was sufficient to attenuate downstream signaling, as C320A-mediated ectopic stabilization of the Hh pathway transcriptional effector Ci was dramatically reduced in response to drug (FIG. 8F compared to E, red). Taken together, these results suggest that alteration of ER homeostasis and induction of the UPR may be a viable option for attenuating aberrant Smo signaling in disease.

### III. Discussion

Herein it has been demonstrated that signaling by active Smo mutants localizing largely to the ER is attenuated under conditions of hyperthermal- or thapsigargin-mediated ER stress. While not limiting the present invention to any particular theory or mechanism, these studies focused on Smo proteins harboring activating C to A mutations in extracellular loop 1 and transmembrane domain 3, and the oncogenic M2 mutation, a W to L alteration in transmembrane domain 7 (Carroll et al., 2012 [31]; Xie et al., 1998 [12]). Each of these mutations likely induces a Smo conformational shift that mimics the Hh-induced active conformation, thereby triggering ligand-independent signaling (Carroll et al., 2012 [31]; Taipale et al., 2000 [8]). Although it is not necessary to understand the mechanism of an invention, it is believed that despite this active conformation, the mutant protein is recognized by the ER as being misfolded, resulting in prolonged ER retention. An exploitation of this phenomenon was attempted to specifically target active Smo mutants, and were successful in attenuating mutant Smo protein stability and downstream signaling, both in vitro and in vivo, by inducing either thermal or chemical ER stress.

Given the significant interest in identifying novel methods of targeting aberrant Hh signaling in disease (Mas & Ruiz i Altaba, 2010 [55]; Robbins et al., 2005 [56]; Scales & de Sauvage, 2009 [57]), it is possible that these findings may have clinical relevance, particularly in cases where disease results from Smo mutation. Previous in vitro studies have demonstrated reduced sensitivity of the oncogenic M2 mutant to small molecule Smo inhibitors (Taipale et al., 2000 [8]). More significantly, acquired resistance to the only FDA approved Smo inhibitor, GDC-0449 (Vismodegib), was recently observed in the clinic (Rudin et al., 2009 [43]). In this case resistance was conferred by a novel Smo mutation that attenuated the ability of the compound to bind to Smo and inhibit its signaling activity (Yauch et al., 2009 [18]). As such, there remains a clinical need for additional methods of targeting the Hh pathway, either in frontline combination therapy or in salvage therapy for relapsed patients who develop resistance to current Smo antagonists. It is not intended that embodiments of the invention be limited to any particular mechanism; however, it is believed that compounds affecting the normal folding environment of the ER might fill this niche by specifically targeting conformationally unstable Smo mutants. Such compounds would offer a significant advantage over Smo-specific small molecules because UPR modulators exploit a cellular process that is distinct from the Hh signaling pathway. As such, their efficacy should be unaltered by acquired Smo mutation.

Admittedly, the current studies were performed on over-expressed Smo, a membrane protein that, when highly expressed, is likely to accumulate in the ER and induce a baseline ER stress response. However, it is not intended that embodiments of the invention be limited to any particular mechanism; however, it is believed that that the observed effects are not due solely to high level Smo expression; over-expressed wild type Smo protein localizing to the ER fraction was not eliminated by the UPR in response to ER stress. As such, without intending that embodiments of the invention be limited to any particular mechanism; however, it is believed that upon inducing ER stress in Smo-expressing cells, wild type Smo protein continues to fold properly, does not engage the UPR and exits the ER through its normal secretory route. Conversely, moderately misfolded active Smo mutants are readily detected by an active UPR, leading to their elimination through ERAD. As such, the observed destabilization and signaling attenuation of active Smo mutants following ER stress induction and UPR engagement results from a molecular mechanism that is specific to mutant Smo protein, an ideal scenario for clinical efficacy. Although the relative abundance of oncogenic SmoM2 protein in a human tumor cell is not known, it is possible that altered conformation and atypical ER exit (Hoffineister et al., 2011 [37]; Taipale et al., 2000 [8]) may result in SmoM2 having an extended ER retention time, thereby sensitizing it to an active ER stress response.

Recent work on Rhodopsin suggests that this may be a recurring theme in G-protein coupled receptor biology. Studies performed using the Drosophila model of autosomal dominant retinitis pigmentosa, which closely mimics the human disease, revealed that sustained ER stress protects against retinal degeneration triggered by ER-retained disease-causing Rhodopsin mutants (Kang & Ryoo, 2009 [58]; Mendes et al., 2009 [59]). These studies demonstrated that, in flies heterozygous for the RhodopsinG69D disease causing mutation, ERAD specifically degraded mutant Rhodopsin, but did not target the wild type protein (Kang & Ryoo, 2009 [58]). Importantly, in these studies, the mutant allele was expressed at endogenous levels off the endogenous promoter (Kang & Ryoo, 2009 [58]; Ryoo et al., 2007 [44]).

Based upon the results presented herein, it is probable that the UPR holds promise to rapidly expand the cache of small molecules available for treatment of Hh-dependent cancer. Notably, a number of ER stress and UPR modulating compounds, including a thapsigargin-derived prodrug, are either approved or currently being evaluated for clinical use in treating cancers such as leukemia, lymphoma, multiple myeloma and prostate cancer (Denmeade et al., 2012 [60]; Li et al., 2011 [16]). As such, if future studies reveal UPR modulating compounds to be efficacious in targeting Smo-driven malignancies, the ability to translate these findings to the clinic may be expedited. This could prove to be a significant advantage over novel Hh pathway specific small molecules that are not yet approved for clinical use.

Thus, specific compositions and methods of use of small molecule unfolded protein response modulators to treat tumors with active sonic hedgehog (SHH) signaling due to smoothened (SMO) mutation have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### Experimental Procedures

### IV. Functional Assays and Biochemical Analyses

Reporter assays were performed as described (Carroll et al., 2012 [31]), with the following minor modifications. For rescue experiments, ˜1.5 e⁶ C18 cells were transfected with 100 ng ptcΔ136-luciferase, 10 ng pAc-renilla, 20 ng smo 5′UTR dsRNA, 100 ng pAc-hh or empty vector control, and 20 ng of the indicated wild type or mutant pAc-smo construct (Carroll et al., 2012 [31]; Chen et al., 1999 [48]; Ogden et al., 2006 [61]). For dominant activity assays, 20 ng of the indicated mycsmo expression vector was expressed in the absence of Hh, and reporter activity assessed as described (Carroll et al., 2012 [31]). For all experiments, cells were transfected at 25° C., and allowed to recover for 24 hours prior to shifting to 22° C. or 29° C. Reporter activity was assessed 24 hours post temperature shift and normalized to the Renilla transfection control. Reporter assays were performed at least two times in duplicate, and all data pooled. Reporter activity is shown as percent activity relative to the control Hh response for each temperature, arbitrarily set to 100%. Error bars represent standard error of the mean.

For protein stability analysis in Drosophila cells, ˜5×10⁶ C18 cells were transfected with 5 ug of wild type or mutant pAc-smo expression vector using Lipofectamine2000 transfection reagent (Invitrogen). Cells were transfected at 25° C., and allowed to recover for 24 hours before shifting to 22° C. or 29° C. Whole cell lysates were prepared 24 hours post temperature shift using SDS lysis buffer (2% SDS, 4% glycerol, 40mM Tris-HCL, pH 6.8, 0.5 mM DTT, and 1× protease inhibitor cocktail (Roche)). Cell extracts were sheared by passing 5 times through a 26 gauge syringe. Equal amounts of total protein were analyzed by SDS-PAGE and western blot using anti-Myc (Roche) and anti-Kinesin (Cytoskeleton) antibodies.

For protein stability analysis in mammalian cells, ˜1×10⁶NIH3T3 cells were transfected with 2 ug of wild type or mutant pcDNA3.1-amino expression vector (Carroll et al., 2012 [31]). For temperature sensitivity analysis, transfected cells were maintained at 37° C. for ˜44 hours, and then shifted to 40° C. for 4 hours prior to lysis. Whole-cell lysates were prepared in modified RIPA buffer (50 mM Tris-HCl, pH 7.4, 150 mM NaCl, 0.25% deoxycholic acid, 1% NP-40, 1 mM EDTA, 0.1% SDS, 0.5 mM DTT, and 1× PIC (Roche)) by rocking for 30 minutes at 4° C. as described (Nachtergaele et al., 2012 [62]). Cell extracts were cleared by centrifuging at 16,000×g at 4° C. for 45 minutes. Supernatants were collected and analyzed by SDS-Page and western blot using anti-GADD153/CHOP (B-3, SCBT) anti-Smo (E5, SCBT) and anti-tubulin (Cell Signaling) antibodies. For thapsigargin sensitivity analysis, NIH3T3 cells expressing wild type or mutant mSmo protein at 37° C. were treated with vehicle (ethanol) or 1 uM thapsigargin (Sigma) in DMEM containing 0.5% fetal calf serum for 4 hrs prior to lysis. Whole-cell lysates were prepared in modified RIPA buffer and analyzed as above.

For glycosylation analysis cell lysates were prepared from C18 or NIH3T3 cells transfected with wild type or mutant smo expression vectors, as described above. C18 cell lysates were prepared in NP-40 lysis buffer (1% NP-40, 150 mM NaCl, 50 mM Tris, 50 mM NaF, 0.5 mM DTT, and 1× PIC (Roche), pH 8.0), and centrifuged for 10 minutes at 2000×g. NIH3T3 cells were lysed using modified RIPA as described above. Supernatants were treated with PNGase, EndoH, O-glycosidase or λ phosphatase (NEB) per manufacturer's instructions for 2 hours at room temperature, and analyzed by SDS-PAGE and western blot as described above.

V. Quantitative rtPCR (qPCR) Analysis

To assess gli1 and smo expression, NIH3T3 cells were plated as above and transfected using Fugene 6 (Promega). Approximately 30 hours post transfection, culture media was replaced with complete media containing 250 nM Thapsigargin or vehicle (ethanol) control. RNA was extracted using RNeasy kit (Qiagen) ˜16 hours after media exchange. cDNA was synthesized from 5 ug of RNA using SuperScript III first strand synthesis system (Invitrogen). qPCR reactions were performed on cDNA diluted 1:10 using SYBR Green PCR master mix (Applied Biosystems). GAPDH was used as a reference gene and results were analyzed using a standard 2^{-ααCt} method (Livak & Schmittgen, 2001 [63]). The following gene specific primers were used:

Gli1-qPCR-f:
(SEQ ID NO: 1)
5′-GGTCTCGGGGTCTCAAACTGC
Gli1-qPCR-r:
(SEQ ID NO: 2)
5′-CGGCTGACTGTGTAAGCAGAG
mSmo-qPCR-f:
(SEQ ID NO: 3)
5′-CGCCAAGGCCTTCTCTAAGCG
mSmo-qPCR-r:
(SEQ ID NO: 4)
5′-CCTCTGCCTGGGCTCAGCAT
mGAPDH-qPCR-f:
(SEQ ID NO: 5)
5′-GTGGTGAAGCAGGCATCTGA
mGAPDH-qPCR-r:
(SEQ ID NO: 6)
5′-GCCATGTAGGCCATGAGGTC

qPCR analysis was performed three times in triplicate, and all data pooled. Error bars indicate standard error of the mean.

### VI. Fly Crosses

Fly stocks were maintained at 18° C. on Jazz mix agarose (Fisher). Crosses were performed at 22° C. or 29° C. as indicated. UAS-myc-smo, UAS-myc-smoC320A and UAS-mycsmoC339A (Carroll et al., 2012 [31]) were expressed under control of MS1096-Gal4. UAS-Xbp1-GFP (Ryon et al., 2007 [44]) was expressed under control of sgs3-Gal4. GFP expression was examined in salivary glands dissected from 3^{rd} instar larvae. Multiple salivary glands were examined across two independent crosses, and representative samples shown. For wing analyses, crosses were performed at least twice, and multiple progeny analyzed. Representative wings from adult flies were mounted on glass slides using DPX mounting media, and imaged on a Zeiss Sterni 2000-C11 microscope with a Zeiss AxioCam ICc3 camera. In cases where wings were severely blistered, the whole fly was imaged. Images were prepared using Photoshop CS4.

For drug treatment, 1 to 24 hour MS1096>myc-smoC320A embryos were collected and transferred to vials containing 2 mL Jazz agarose containing vehicle (ethanol) or thapsigargin (Sigma) at a final concentration of 1 uM. Drug feeding was performed across two separate crosses and multiple progeny analyzed. Representative wings from the resulting adult flies were mounted and imaged as above.

### VII. Immunofluorescence

pAc-myc-smo constructs were expressed in Schneider 2 (S2) cells as described (Carroll et al., 2012 [31]). For temperature sensitivity analyses cells were transfected at 25° C. and allowed to recover for 6 hours prior to shifting to 22° C. or 29° C. Fixation, immunostaining and image analysis were performed 48 hours post temperature shift, as previously described (Carroll et al., 2012 [31]). Primary cilium analysis in NIH3T3 cells was performed exactly as described (Carroll et al., 2012 [31]). GRP94 antibody was provided by L. Hendershot (Shen et al., 2002 [64]). Imaginal disc analysis was performed exactly as described (Carroll et al., 2012 [31]). For all indirect immunofluorescence, the indicated primary antibodies were detected using Alexa Fluor (Invitrogen) secondary antibodies conjugated to 488 or 555 fluorophores. Data were obtained using a Zeiss LSM 510 confocal microscope and processed using LSM Image Browser and Photoshop CS4 software. Channels were pseudo-colored as indicated in the text.

### VIII. Initial Testing of Compounds

The four ER stress/UPR modulating compounds listed below (abbreviated and commercial names in parentheses) destabilize oncogenic SmoM2 protein in cultured mouse NIH3T3 cells.
1. 17-N-Allylamino-17-demethoxygeldanamycin (17-AAG, KOS-953,Tanespimycin),
2. 17-Dimethylaminoethylamino-17-demethoxygeldanamycin (17-DMAG, KOS-1022, Alvespimycin),
3. 4-(6,6-dimethyl-4-oxo-3-(trifluoromethyl)-4,5,6,7-tetrahydroindazol-1-yl)-2-((1r,4r)-4-hydroxycyclohexylamino)benzamide (SNX-2112)
4. Eeyarestatin I (EerI).

### IX. Planned/Prophetic Experimental Procedures and Protocols for Theoretical Testing of Compounds

### Testing Unfolded Protein Response (UPR) Modulation for Efficacy Against a Smoothened M2 (SmoM2) Tumor Model

An assessment of whether UPR activation might have efficacy for treating tumors harboring an active Hedgehog pathway due to Smo mutation using three independent animal models will be made. For initial studies, thapsigargin, a compound derived from Thapsia garganica, will be used. Thapsigargin potently induces the UPR in cultured cells and animal models. Secondary studies will involve additional UPR-inducing compounds (HSP-90 inhibitors, Epigallocatechin gallate (EGCG), a GRP78 inhibitor and G-202, a thapsigargin derived pro-drug). While not limiting the current invention, it should be noted that thapsigargin by itself is not an ideal compound to use due to associated toxicity. Thapsigargin has only been used in a couple of animal studies in the literature and is employed as model compound. Dose limiting toxicity in mice will be determined, then effects of thapsigargin on M2-driven tumors only will be assessed as proof-of-principle initial studies. For follow-on studies, the thapsigargin pro-drug G-202 will be employed, as it demonstrates significantly reduced in vivo toxicity.

1. Test against SMOM2-driven medulloblastoma model (in collaboration with Young-Goo Han, St. Jude Children's Research Hospital Department of Developmental Neurobiology): Transgenic mice expressing SmoM2 under control of GFAP/Cre. Mice that express SmoM2 develop medulloblastomas in utero, and typically die from their tumors by age p15. Thapsigargin will be administered at a concentration of 0.5 mg/kg to p11-p12 neonates by intraperitoneal injection. Mice will be sacrificed 8-10 hours after drug administration and brain tissue processed for immunohistochemical analysis. Due to the high toxicity of thapsigargin, we will not perforin multiple dosings. SmoM2 protein stability will be assessed in tumor sections from treated animals, and compared to sections from vehicle (DMSO) treated animals. Levels of SmoM2 protein in tumors will be examined. Given that UPR induction by thapsigargin triggers degradation of oncogenic SmoM2 protein in vitro, it is hypothesized that if the compound has efficacy in this tumor model, lower levels of SmoM2 protein in tumors from treated animals will be detected.

2. Test against SMOM2 driven rhabdomyosarcoma model (in collaboration with Mark Hatley in the St. Jude Children's Research Hospital Department of Oncology): In order to assess the in vivo anti-SinoM2 effects of Thapsigargin, a transformed human myoblast cell line, HSMM, will be used. The Hatley lab has immortalized human skeletal muscle myoblasts with transduction with viruses overexpressing SV40 Large T and small t antigen and the catalytic subunit of telomerase, hTERT. These immortalized human skeletal muscle myoblasts, HSMM-TH cells, provide a platform to test the transforming potential of oncogenes. Transduction of the HSMM-TH cells oncogenic HRAS-G12V results in transformation and the formation of tumors resembling pediatric embryonal rhabdomyosarcoma when injected into immuno compromised mice. The Hatley lab has transduced the HSMM-TH myoblasts with a virus expressing the oncogenic SMOM2 allele (HSMM-THS cells) that results in constitutive activation of the Shh pathway. Xenografts in immunocompromised mice of the HSMM-THS will allow for in vivo validation of the effects of Thapsigargin on Hh-driven tumors. Immunocompromised mice will be injected with the HSMM-THS cells and tumors will be allowed to form over a period of two weeks. After the tumors are evident, one group of animals will be injected with Thapsigargin and the other will be injected with a vehicle control by a blinded investigator. A blinded researcher will monitor the tumor volume over time. When the tumors reach the tumor volume limits, all the animals will be sacrificed and weighed. The xenografted tumors will be dissected and weighed. The effect of Thapsigarin on tumor volume and tumor mass will be reported. The dissected tumor will be divided with aliquots flash frozen in liquid nitrogen for gene expression and protein analysis, and a portion will be fixed in 4% parafonnaldehyde for histology.

3. Xenograft of Sonic Hedgehog subtype medulloblastoma in nude mice (in collaboration with Chris Morton in the St. Jude Chilren's Research Hospital Department of Surgery). Growing SHH subtype medulloblastoma cells in nude mice is in progress. Once tumors develop, mice with will be treated with thapsigargin and/or additional compounds affecting the UPR, and examined for tumor regression by monitoring tumor volume. Tumor tissue will be harvested and assessed for Smo protein expression.

If effects on SmoM2 protein stability and/or tumor growth in any of the models above are observed, additional drugs and/or chemical modification of Thapsigargin to reduce toxicity will be targeted and considered part of the current invention.

### REFERENCES:

1. Harding, H. P., Zhang, Y., and Ron, D. (1999) Protein translation and folding are coupled by an endoplasmic-reticulum-resident kinase, Nature 397(6716), 271-274.
2. McCracken, A. A. and Brodsky, J. L. (1996) Assembly of ER-associated protein degradation in vitro: dependence on cytosol, calnexin, and ATP, The Journal of Cell Biology 132(3), 291-298.
3. Tirasophon, W. et al. (2000) The endoribonuclease activity of mammalian IRE1 autoregulates its mRNA and is required for the unfolded protein response, Genes Dev. 14(21), 2725-2736.
4. Walter, P. and Ron, D. (2011) The Unfolded Protein Response: From Stress Pathway to Homeostatic Regulation, Science 334(6059), 1081-1086.
5. Yoshida, H. et al. (1998) Identification of the cis-Acting Endoplasmic Reticulum Stress Response Element Responsible for Transcriptional Induction of Mammalian Glucose-regulated Proteins, J. Biol. Chem. 273(50), 33741-33749.
6. Taipale, J. et al. (2002) Patched acts catalytically to suppress the activity of Smoothened, Nature 418(6900), 892-896.
7. Ruiz-Gómez, A. et al. (2007) The cell biology of Smo signalling and its relationships with GPCRs, Biochimica et Biophysica Acta (BBA)-Biomembranes 1768(4), 901-912.
8. Taipale, J. et al. (2000) Effects of oncogenic mutations in Smoothened and Patched can be reversed by cyclopamine, Nature 406(6799), 1005-1009.
9. Corbit, K. C. et al. (2005) Vertebrate Smoothened functions at the primary cilium, Nature 437(7061), 1018-1021.
10. Lum, L. et al. (2003) Hedgehog Signal Transduction via Smoothened Association with a Cytoplasmic Complex Scaffolded by the Atypical Kinesin, Costal-2, Mol. Cell 12(5), 1261-1274.
11. Endoh-Yamagami, S. et al. (2009) The Mammalian Cos2 Homolog Kif7 Plays an Essential Role in Modulating Hh Signal Transduction during Development, Current biology: CB 19(15), 1320-1326.
12. Xie, J. et al. (1998) Activating Smoothened mutations in sporadic basal-cell carcinoma, Nature 391(6662), 90-92.
13. Backer, J. et al. (2009) Chaperone-targeting cytotoxin and endoplasmic reticulum stress-inducing drug synergize to kill cancer cells, Neoplasia 11(11), 1165-1173.
14. Ng, J. M. Y. and Curran, T. (2011) The Hedgehog's tale: developing strategies for targeting cancer, Nat. Rev. Cancer 11(7), 493-501.
15. Dijkgraaf, G. J. P. et al. (2011) Small Molecule Inhibition of GDC-0449 Refractory Smoothened Mutants and Downstream Mechanisms of Drug Resistance, Cancer Res. 71(2), 435-444.
16. Li, X., Zhang, K., and Li, Z. (2011) Unfolded protein response in cancer: the Physician's perspective, J. Hematol. Oncol. J Hematol Oncol 4(1), 8-17.
17. Olive, K. P. and Tuveson, D. “Hedgehog Pathway Inhibitors,” United States Patent Application 20120020876 application Ser. No. 13/144992, filed January 22, 2010. (Published Jan. 26, 2012).
18. Yauch, R. L. et al. (2009) Smoothened Mutation Confers Resistance to a Hedgehog Pathway Inhibitor in Medulloblastoma, Science 326(5952), 572-574.
19. Barakat, M. T., Humke, E. W., and Scott, M. P. (2010) Learning from Jekyll to control Hyde: Hedgehog signaling in development and cancer, Trends Mol. Med. 16(8), 337-348.
20. Ingham, P. W. and McMahon, A. P. (2001) Hedgehog signaling in animal development: paradigms and principles, Genes Dev. 15(23), 3059-3087.
21. Jiang, J. and Hui, C.-c. (2008) Hedgehog Signaling in Development and Cancer, Dev. Cell 15(6), 801-812.
22. Berman, D. M. et al. (2003) Widespread requirement for Hedgehog ligand stimulation in growth of digestive tract tumours, Nature 425(6960), 846-851.
23. Fan, H. et al. (1997) Induction of basal cell carcinoma features in transgenic human skin expressing Sonic Hedgehog, Nat. Med. 3(7), 788-792.
24. Goodrich, L. V. et al. (1997) Altered neural cell fates and medulloblastoma in mouse patched mutants, Science 277(5329), 1109-1113.
25. Karhadkar, S. S. et al. (2004) Hedgehog signalling in prostate regeneration, neoplasia and metastasis, Nature 431(7009), 707-712.
26. Watkins, D. N. et al. (2003) Hedgehog signalling within airway epithelial progenitors and in small-cell lung cancer, Nature 422(6929), 313-317.
27. Yuan, Z. et al. (2006) Frequent requirement of hedgehog signaling in non-small cell lung carcinoma, Oncogene 26(7), 1046-1055.
28. Alcedo, J. et al. (1996) The Drosophila smoothened Gene Encodes a Seven-Pass Membrane Protein, a Putative Receptor for the Hedgehog Signal, Cell 86(2), 221-232.
29. van den Heuvel, M. and Ingham, P. W. (1996) Smoothened encodes a receptor-like serpentine protein required for hedgehog signalling, Nat. Rev. Mol. Cell Biol. 382(6591), 547-551.
30. Lam, C. W. et al. (1999) A frequent activated smoothened mutation in sporadic basal cell carcinomas, Oncogene 18(3), 833-836.
31. Carroll, C. E. et al. (2012) The extracellular loops of Smoothened play a regulatory role in control of Hedgehog pathway activation, Development 139(3), 612-621.
32. Cook, J. V. et al. (1996) A disulfide bonding interaction role for cysteines in the extracellular domain of the thyrotropin-releasing hormone receptor, Endocrinology 137(7), 2851-2858.
33. Karnik, S. S. et al. (1988) Cysteine residues 110 and 187 are essential for the formation of correct structure in bovine rhodopsin, Proc. Natl. Acad. Sci. U. S. A. 85(22), 8459-8463.
34. Moro, S., Hoffmann, C., and Jacobson, K. A. (1999) Role of the Extracellular Loops of G Protein-Coupled Receptors in Ligand Recognition: A Molecular Modeling Study of the Human P2Y1 Receptor, Biochemistry 38(12), 3498-3507.
35. Chen, J. K. et al. (2002) Inhibition of Hedgehog signaling by direct binding of cyclopamine to Smoothened, Genes Dev. 16(21), 2743-2748.
36. Incardona, J. P., Gruenberg, J., and Roelink, H. (2002) Sonic Hedgehog Induces the Segregation of Patched and Smoothened in Endosomes, Current biology: CB 12(12), 983-995.
37. Hoffineister, H. et al. (2011) Polycystin-2 takes different routes to the somatic and ciliary plasma membrane, The Journal of Cell Biology 192(4), 631-645.
38. Rohatgi, R. et al. (2009) Hedgehog signal transduction by Smoothened: Pharmacologic evidence for a 2-step activation process, Proc. Natl. Acad. Sci. U. S. A. 106(9), 3196-3201.
39. Wong, S. Y. et al. (2009) Primary cilia can both mediate and suppress Hedgehog pathway-dependent tumorigenesis, Nat. Med. 15(9), 1055-1061.
40. Han, Y.-G et al. (2008) Hedgehog signaling and primary cilia are required for the formation of adult neural stem cells, Nat. Neurosci. 11(3), 277-284.
41. Hetz, C. (2012) The unfolded protein response: controlling cell fate decisions under ER stress and beyond, Nat. Rev. Mol. Cell Biol. 13(2), 89-102.
42. Liu, Y. and Ye, Y (2011) Proteostasis regulation at the endoplasmic reticulum: a new perturbation site for targeted cancer therapy, Cell Res. 21(6), 867-883.
43. Rudin, C. M. et al. (2009) Treatment of Medulloblastoma with Hedgehog Pathway Inhibitor GDC-0449, N. Engl. J. Med. 361(12), 1173-1178.
44. Ryoo, H. D. et al. (2007) Unfolded protein response in a Drosophila model for retinal degeneration, EMBO J. 26(1), 242-252.
45. Iwawaki, T. et al. (2004) A transgenic mouse model for monitoring endoplasmic reticulum stress, Nat. Med. 10(1), 98-102.
46. Duffy, J. B. (2002) GAL4 system in drosophila: A fly geneticist's swiss army knife, Genesis 34(1-2), 1-15.
47. Aza-Blanc, P. et al. (1997) Proteolysis That Is Inhibited by Hedgehog Targets Cubitus interruptus Protein to the Nucleus and Converts It to a Repressor, Cell 89(7), 1043-1053.
48. Chen, C.-H. et al. (1999) Nuclear Trafficking of Cubitus interruptus in the Transcriptional Regulation of Hedgehog Target Gene Expression, Cell 98(3), 305-316.
49. Alcedo, J., Zou, Y, and Noll, M. (2000) Posttranscriptional Regulation of Smoothened Is Part of a Self-Correcting Mechanism in the Hedgehog Signaling System, Mol. Cell 6(2), 457-465.
50. Denef, N. et al. (2000) Hedgehog Induces Opposite Changes in Turnover and Subcellular Localization of Patched and Smoothened, Cell 102(4), 521-531.
51. Ingham, P. W. et al. (2000) Patched represses the Hedgehog signalling pathway by promoting modification of the Smoothened protein, Current biology: CB 10(20), 1315-1318.
52. Casso, D. J. et al. (2005) Drosophila Signal Peptide Peptidase Is an Essential Protease for Larval Development, Genetics 170(1), 139-148.
53. Xu, X. et al. (2011) Hyperthermia Induces the ER Stress Pathway, PLoS One 6(8), c23740.
54. Li, M. et al. (2000) ATF6 as a Transcription Activator of the Endoplasmic Reticulum Stress Element: Thapsigargin Stress-Induced Changes and Synergistic Interactions with NF-Y and YY1, Mol. Cell. Biol. 20(14), 5096-5106.
55. Mas, C. and Ruiz i Altaba, A. (2010) Small molecule modulation of HH-GLI signaling: Current leads, trials and tribulations, Biochem. Pharmacol. 80(5), 712-723.
56. Robbins, D. J. et al. (2005) Inhibitors of the Hedgehog Signal Transduction Pathway, Current Cancer Therapy Reviews 1(3), 277-288.
57. Scales, S. J. and de Sauvage, F. J. (2009) Mechanisms of Hedgehog pathway activation in cancer and implications for therapy, Trends Pharmacol. Sci. 30(6), 303-312.
58. Kang, M.-J. and Ryoo, H. D. (2009) Suppression of retinal degeneration in Drosophila by stimulation of ER-associated degradation, Proc. Natl. Acad. Sci. U. S. A. 106(40), 17043-17048.
59. Mendes, C. S. et al. (2009) ER stress protects from retinal degeneration, EMBO J. 28(9), 1296-1307.
60. Denmeade, S. R. et al. (2012) Engineering a Prostate-Specific Membrane Antigen-Activated Tumor Endothelial Cell Prodrug for Cancer Therapy, Science Translational Medicine 4(140), 140ra186.
61. Ogden, S. K. et al. (2006) Smoothened Regulates Activator and Repressor Functions of Hedgehog Signaling via Two Distinct Mechanisms, J. Biol. Chem. 281(11), 7237-7243.
62. Nachtergaele, S. et al. (2012) Oxysterols are allosteric activators of the oncoprotein Smoothened, Nat. Chem. Biol. 8(2), 211-220.
63. Livak, K. J. and Schmittgen, T. D. (2001) Analysis of Relative Gene Expression Data Using Real-Time Quantitative PCR and the 2-ΔΔCT Method, Methods 25(4), 402-408.
64. Shen, Y., Meunier, L., and Hendershot, L. M. (2002) Identification and Characterization of a Novel Endoplasmic Reticulum (ER) DnaJ Homologue, Which Stimulates ATPase Activity of BiP in Vitro and Is Induced by ER Stress, J. Biol. Chem. 277(18), 15947-15956.

We claim:

1. A method of treatment, comprising:
administering an endoplasmic reticulum stressor compound to a subject having a cancer, wherein said cancer comprises a smoothened mutation.

2. The method of claim 1, wherein said cancer cells comprise Sonic Hedgehog-driven tumors.

3. The method of claim 1, wherein said cancer is selected form the group of cancers consisting of basal cell carcinoma, medulloblastoma, rhabdomyosarcoma, multiple myeloma and prostate cancer.

4. The method of claim 1, wherein said treatment comprises administration of a drug selected from the group consisting of: 17-N-allylamino-17-demethoxygeldanamycin, 17-dimethylaminoethylamino-17-demethoxygeldanamycin, 4-(6,6-dimethyl-4-oxo-3-(trifluoromethyl)-4,5,6,7-tetrahydroindazol-1-yl)-2-((1r,4r)-4-hydroxycyclohexylamino)benzamide, and eeyarestatin I.

5. The method of claim 1, wherein said treatment comprises administration of a drug selected from the group consisting of: NPI-0052, carfilzomib, PS-341, CEP-18770, retaspimycin, PU-H71, versipelostatin, (−)-epigallocatechin gallate, epidermal growth factor-subA, irestatins, and delta(9)-tetrahydrocannabinol.

6. A method of treatment, comprising:
administering an endoplasmic reticulum stressor compound to a subject having a cancer that has become resistant to previous treatment with a hedgehog inhibitor, wherein said cancer comprises a smoothened mutation.

7. The method of claim 6, wherein said cancer is selected from the group of cancers consisting of basal cell carcinoma, rhabdomyosarcoma, multiple myeloma and prostate cancer.

8. The method of claim 6, wherein said treatment comprises administration of a drug selected from the group consisting of: 17-N-allylamino-17-demethoxygeldanamycin, 17-dimethylamino ethyl amino-17-demethoxygeldanamycin, 4-(6,6-dimethyl-4-oxo-3-(trifluoromethyl)-4,5,6,7-tetrahydroindazol-1-yl)-2-((1r,4r)-4-hydroxycyclohexylamino)benzamide, and eeyarestatin I.

9. The method of claim 6, wherein said treatment comprises administration of a drug selected from the group consisting of: NPI-0052, carfilzomib, PS-341, CEP-18770, retaspimycin, PU-H71, versipelostatin, (−)-epigallocatechin gallate, epidermal growth factor-subA, irestatins, and delta(9)-tetrahydrocannabinol.

10. A method of treatment comprising:
a) providing a sample of cancer from a subject;
b) testing said sample to determine whether said cancer has a smoothened mutation and whether tumor cells are sensitive to ER stressors ex vivo; and
c) treating said subject with an endoplasmic reticulum stressor compound where said cancer comprises a smoothened mutation.

11. The method of claim 10, wherein said cancer is selected from the group of cancers consisting of basal cell carcinoma, rhabdomyosarcoma, medulloblastoma, multiple myeloma and prostate cancer.

12. The method of claim 10, wherein said treatment comprises administration of a drug selected from the group consisting of: 17-N-allylamino-17-demethoxygeldanamycin, 17-dimethylaminoethylamino-17-demethoxygeldanamycin, 4-(6,6-dimethyl-4-oxo-3-(trifluoromethyl)-4,5,6,7-tetrahydroindazol-1-yl)-2-((1r,4r)-4-hydroxycyclohexylamino)benzamide, and eeyarestatin I.

13. The method of claim 10, wherein said treatment comprises administration of a drug selected from the group consisting of: NPI-0052, carfilzomib, PS-341, CEP-18770, retaspimycin, PU-H71, versipelostatin, (−)-epigallocatechin gallate, epidermal growth factor-subA, irestatins, and delta(9)-tetrahydrocannabinol.

14. A method of treatment, comprising:
administering an unfolded protein response inducing compound to a subject having a cancer, wherein said cancer comprises a smoothened mutation.

15. The method of claim 14, wherein said cancer cells comprise Sonic Hedgehog-driven tumors.

16. The method of claim 14, wherein said cancer is selected form the group of cancers consisting of basal cell carcinoma, rhabdomyosarcoma, medulloblastoma, multiple myeloma and prostate cancer.

17. The method of claim 14, wherein said treatment comprises administration of a drug selected from the group consisting of: 17-N-allylamino-17-demethoxygeldanamycin, 17-dimethylaminoethylamino-17-demethoxygeldanamycin, 4-(6,6-dimethyl-4-oxo-3-(trifluoromethyl)-4,5,6,7-tetrahydroindazol-1-yl)-2-((1r,4r)-4-hydroxycyclohexylamino)benzamide, and eeyarestatin I.

18. The method of claim 14, wherein said treatment comprises administration of a drug selected from the group consisting of: NPI-0052, carfilzomib, PS-341, CEP-18770, retaspimycin, PU-H71, versipelostatin, (−)-epigallocatechin gallate, epidermal growth factor-subA, irestatins, and delta(9)-tetrahydrocannabinol.

19. A method of treatment, comprising:
administering an unfolded protein response (UPR) inducing compound to a subject having a cancer that has become resistant to previous treatment with a hedgehog inhibitor, wherein said cancer comprises a smoothened (SMO) mutation.

20. The method of claim 19, wherein said cancer is selected form the group of cancers consisting of basal cell carcinoma, leukemia, lymphoma, multiple myeloma and prostate cancer.

21. The method of claim 19, wherein said treatment comprises administration of a drug selected from the group consisting of: 17-N-allylamino-17-demethoxygeldanamycin, 17-dimethylaminoethylamino-17-demethoxygeldanamycin, 4-(6,6-dimethyl-4-oxo-3-(trifluoromethyl)-4,5,6,7-tetrahydroindazol-1-yl)-2-((1r,4r)-4-hydroxycyclohexylamino)benzamide, and eeyarestatin I.

22. The method of claim 19, wherein said treatment comprises administration of a drug selected from the group consisting of: NPI-0052, carfilzomib, PS-341, CEP-18770, retaspimycin, PU-H71, versipelostatin, (−)-epigallocatechin gallate, epidermal growth factor-subA, irestatins, and delta(9)-tetrahydrocannabinol.

23. A method of treatment comprising:
a) providing a sample of cancer from a subject;
b) testing said sample to determine whether said cancer has a smoothened (SMO) mutation; and
c) treating said subject with an unfolded protein response (UPR) inducing compound where said cancer comprises a smoothened (SMO) mutation.

24. The method of claim 23, wherein said cancer is selected form the group of cancers consisting of basal cell carcinoma, leukemia, lymphoma, multiple myeloma and prostate cancer.

25. The method of claim 23, wherein said treatment comprises administration of a drug selected from the group consisting of: 17-N-allylamino-17-demethoxygeldanamycin, 17-dimethylaminoethylamino-17-demethoxygeldanamycin, 4-(6,6-dimethyl-4-oxo-3-(trifluoromethyl)-4,5,6,7-tetrahydroindazol-1-yl)-2-((1r,4r)-4-hydroxycyclohexylamino)benzamide, and eeyarestatin I.

26. The method of claim 23, wherein said treatment comprises administration of a drug selected from the group consisting of: NPI-0052, carfilzoinib, PS-341, CEP-18770, retaspimycin, PU-H71, versipelostatin, (−)-epigallocatechin gallate, epidermal growth factor-subA, irestatins, and delta(9)-tetrahydrocannabinol.
